(19) United States
(12) Patent Application Publication
(10) Pub. No.: US 2022/0118098 A1
(43) Pub. Date: Apr. 21, 2022

(54) MULTI-CONJUGATES COMPRISING A MONO-SUBSTITUTED HOMO-BIVALENT LINKER

(71) Applicant: MPEG LA, L.L.C., Chevy Chase, MD (US)

(72) Inventors: Jonathan Miles Brown, Silver Spring, MD (US); Kristin K. H. Neuman, Larchmont, NY (US)

(21) Appl. No.: 17/503,053

(22) Filed: Oct. 15, 2021

Related U.S. Application Data
(60) Provisional application No. 63/093,085, filed on Oct. 16, 2020.

(51) Int. Cl. A61K 47/54; A61K 45/06; A61K 47/64

(52) U.S. Cl. CPC A61K 47/545; A61K 47/64; A61K 47/542; A61K 45/06

(57) ABSTRACT

Various embodiments provide a homo-bivalent covalent linker substituted on one end by a substituent X, wherein X comprises a biological moiety other than a nucleic acid.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to novel synthetic intermediates comprising mono-substituted homo-bivalent linkers, and use of the intermediates in the synthesis of multi-conjugates for use in modulating gene expression, biological research, treating or preventing medical conditions, or to produce new or altered phenotypes.

### BACKGROUND

Bioconjugates comprise covalent linkages of at least two molecules, at least one of which is a biomolecule. Biomolecules have a variety of functions, such as in labeling, imaging, and tracking molecular and cellular events, delivering drugs to targeted cells, and as diagnostic or therapeutic agents. Nonlimiting examples of bioconjugates include the coupling of a small molecule (e.g., biotin) to a protein, protein-protein conjugates (e.g., an antibody coupled to an enzyme), antibody drug conjugates (ADCs) (e.g., a monoclonal antibody conjugated to a cytotoxic small molecule), radio-immunoconjugates (e.g., a monoclonal antibody conjugated to a chelating agent), vaccines (e.g., haptens conjugated to carrier proteins), antibodies conjugated to nanoparticles and non-cytotoxic drugs (e.g., peptides), biomolecules conjugated to elements or derivatives thereof (e.g., TGF-β conjugated to iron oxide nanoparticles).

Bioconjugates pose developmental and manufacturing challenges, many stemming from the process of forming covalent linkages (conjugation) at sufficient yield and purity. For example, the use of a homo-bivalent linker to form a hetero-dimer of substituents A and B, under some conditions, will produce a mixture of homo-dimer and hetero-dimer species, from which, isolation of the desired hetero-dimer may be difficult if the substituents A and B are similar in size and/or charge. This problem may be addressed by the use of a hetero-bivalent linker to form the hetero-dimer of substituents A and B; yet, new problems arise if, for example, a cleavable linkage is desired and a hetero-bivalent linker with a desirable cleavage profile is unavailable; or if, for example, a further conjugation reaction is desired (e.g., to form a multi-conjugate of three substituents, A, B and C).

There is, therefore, a need for new methods and materials to improve the efficiency of bioconjugate development, synthesis, and cost effective scale-up for commercial use.

### SUMMARY OF THE DISCLOSURE

The present disclosure relates to compounds representing a new class of synthetic intermediates, methods of using the synthetic intermediates to synthesize multi-conjugates, new forms of multi-conjugates, and methods of using the multi-conjugates, for example in reducing gene expression, biological research, treating or preventing medical conditions, and/or to produce new or altered phenotypes.

The disclosure provides for a synthetic intermediate comprising a homo-bivalent covalent linker substituted on one end by a substituent X, while remaining unsubstituted on the other end (a “mono-substituted covalent linker”), and wherein X comprises a biological moiety other than a nucleic acid. The disclosure is applicable to all types of homo-bivalent covalent linkers, cleavable or noncleavable, including but not limited to the examples of homo-bivalent covalent linkers provided throughout the disclosure. The disclosure is applicable to all types of non-nucleic acid biological moieties as the substituent, including but not limited to the examples of non-nucleic acid substituents provided throughout the disclosure.

The disclosure provides methods for synthesizing a mono-substituted covalent linker comprising the steps of coupling a homo-bivalent covalent linker to a substituent X, wherein X is a biological moiety other than a nucleic acid, under reaction conditions that substantially favor the formation of the mono-substituted covalent linker and substantially prevent dimerization of the substituent X.

The disclosure also provides for multi-conjugates comprising a first substituent X, which comprises a biological moiety other than a nucleic acid and a second substituent Y that is the same or different from X, wherein X and Y are joined by a homo-bivalent covalent linker. The disclosure is applicable to all types of homo-bivalent covalent linkers, cleavable or noncleavable, including but not limited to the examples of homo-bivalent covalent linkers provided throughout the disclosure; and further, to all types of biological moieties as the substituents, non-nucleic in the case of X, and nucleic or non-nucleic in the case of Y, including but not limited to the examples of substituents provided throughout the disclosure.

The disclosure provides methods for synthesizing multi-conjugates comprising a first substituent X, which comprises a biological moiety other than a nucleic acid and a second substituent Y that is the same or different from X, wherein X and Y are joined by a homo-bivalent covalent linker under reaction conditions that substantially favor formation of the X—Y dimer and substantially prevent formation of the X—X dimer and the Y—Y dimer.

The disclosure also provides for multi-conjugates comprising biologically active substituents X, Y and Z, which may be the same or different, each joined to another by a covalent linker. The disclosure is applicable to all types of homo-bivalent covalent linkers, cleavable or noncleavable, including but not limited to the examples of homo-bivalent covalent linkers provided throughout the disclosure; and further, to all types of biological moieties as the substituents, X, Y, and Z, including, but not limited to, the examples of substituents provided throughout the disclosure.

The disclosure also provides methods for synthesizing a multi-conjugate comprising biologically active substituents X, Y and Z, which may be the same or different, each joined to another by a covalent linker.

The disclosure also provides methods of using the disclosed synthetic intermediates and multi-conjugates in modulating gene expression, biological research, treating or preventing medical conditions, and/or to produce new or altered genotypes or phenotypes.

In an embodiment, the disclosure provides a compound (a synthetic intermediate) comprising a homo-bivalent covalent linker substituted on one end by a substituent X, wherein X comprises a biological moiety other than a nucleic acid, wherein the other end of the homo-bivalent linker is unsubstituted, and wherein the compound is at least 75% pure.

In an embodiment, the compound comprises Structure 1:

X—R1-R2-A-R3-B (Structure 1)

wherein:
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
A is a group comprising the reaction product of a first nucleophile and a first electrophile;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide; and
B is a group comprising a second nucleophile or a second electrophile, wherein the second nucleophile is the same as the first nucleophile, and the second electrophile is the same as the first electrophile.

In an embodiment, the spacer group R2 comprises a C₂-C₁₀ alkyl, C₂-C₁₀ alkoxy, or C₁-C₁₀ aryl, or is absent.

In an embodiment, the first nucleophile and first electrophile of the group A comprise (i) a thiol and a maleimide, optionally wherein the reaction product of the thiol and maleimide is a derivative of succinamic acid (such as, when one of the maleimide rings is opened); (ii) a thiol and a vinylsulfone; (iii) a thiol and a pyridyldisulfide; (iv) a thiol and an iodoacetamide; (v) a thiol and an acrylate; (vi) an azide and an alkyne; or (vii) an amine and a carboxyl.

In an embodiment, A is a group comprising the reaction product of a thiol and a maleimide, optionally wherein the reaction product of the thiol and maleimide is a derivative of succinamic acid.

In an embodiment, the R3 group comprises a thiopropionate, a disulfide, an oligonucleotide, or an oligopeptide.

In an embodiment, the compound comprises Structure 2 or a pyrrolidinedione ring-opened derivative thereof, optionally wherein the pyrrolidinedione ring-opened derivative of Structure 2 is a derivative of succinamic acid:

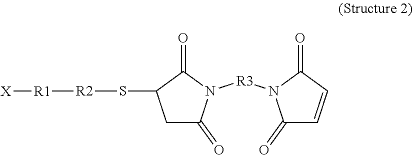

wherein:
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
S is sulfur;
N is nitrogen; and
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide.

In an embodiment, the compound comprises a pyrrolidinedione ring-opened derivative of the compound of Structure 2 that is a derivative of succinamic acid having the following Structure 2a (representing two positional isomers):

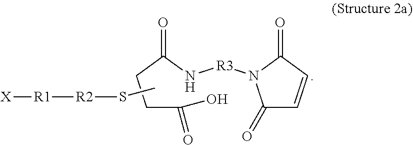

In an embodiment of a compound comprising Structure 2 or Structure 2a, the R2 spacer group comprises a C₂-C₁₀ alkyl, C₂-C₁₀ alkoxy, or C₁-C₁₀ aryl.

In an embodiment of a compound comprising Structure 2 or Structure 2a:
X is a peptide or protein, or a derivative thereof;
R1 and R2 are absent; and
R3 is a group comprising a thiopropionate, disulfide, or oligonucleotide.

In an embodiment of a compound comprising Structure 2 or Structure 2a:
X is an organometallic compound, or a derivative thereof;
R1 is an ester group;
R2 is a spacer group comprising a C₂-C₁₀ alkyl; and
R3 is a group comprising a thiopropionate, a disulfide, an oligonucleotide, or an oligopeptide.

In an embodiment of a compound comprising Structure 2 or Structure 2a:
X is a small molecule, or a derivative thereof;
R1 is an ester group;
R2 is a spacer group comprising a C₂-C₁₀ alkyl; and
R3 is a group comprising a thiopropionate, a disulfide, an oligonucleotide, or an oligopeptide.

In an embodiment of the compound, the homo-bivalent covalent linker comprises a linker that is cleavable under intracellular conditions.

In an embodiment of a compound comprising Structure 1 or Structure 2, the R3 group comprises a linker that is cleavable under intracellular conditions.

In an embodiment of the compound, substituent X is a peptide, protein, lipid, carbohydrate, carboxylic acid, vitamin, steroid, lignin, small molecule, organometallic compound, or a derivative of any of the foregoing.

In an embodiment of the compound, substituent X is a peptide, or a derivative of a peptide. In an embodiment, the peptide comprises the transduction domain of HIV-1TAT protein, or a derivative thereof. In an embodiment, the peptide comprises a Centryrin. In an embodiment, the peptide comprises a constrained peptide. In an embodiment, the peptide comprises a pHLIP peptide.

In an embodiment of the compound, substituent X is an antibody or antibody fragment, or a derivative thereof. In an embodiment, the antibody fragment comprises a single-chain variable fragment, or derivative thereof.

In an embodiment of the compound, the substituent X is a carbohydrate, or a derivative of a carbohydrate.

In an embodiment of the compound, the substituent X is a fatty acid, or a derivative of a fatty acid.

In an embodiment of the compound, the substituent X is a vitamin, or a derivative of a vitamin. In an embodiment, the vitamin is tocopherol or folate, or a derivative thereof.

In an embodiment of the compound, the substituent X is a cholesterol, or a derivative thereof.

In an embodiment of the compound, the substituent X is (2S,2′S)-2,2′-(Carbonyldiimino)dipentanedioic acid (DUPA), or a derivative thereof.

In an embodiment of the compound, the substituent X is anisamide, or a derivative thereof.

In an embodiment of the compound, the substituent X is an organometallic compound, or a derivative thereof. In an embodiment, the organometallic compound is ferrocene, or a derivative thereof.

In an embodiment of the compound, substituent X is a small molecule, or a derivative thereof. In an embodiment, the small molecule is a therapeutic drug molecule. For example, in an embodiment, the small molecule is lenalidomide, or a derivative thereof.

In an embodiment, the compound is at least 80, 85, 90, 95, 96, 97, 98, 99, or 100% pure.

The disclosure provides a method for synthesizing a mono-substituted covalent linker comprising the steps of coupling a homo-bivalent covalent linker to a substituent X, wherein X is a biological moiety other than a nucleic acid, under reaction conditions that substantially favor the formation of the mono-substituted covalent linker and substantially prevent dimerization of the substituent X.

In an embodiment, the method further comprises reacting the homo-bivalent covalent linker with a functionalized substituent X that comprises a functional group reactive with the homo-bivalent covalent linker. In an embodiment of this method, the functionalized substituent X comprises Structure 3:

X—R1-R2-A′ (Structure 3); and

the homo-bivalent covalent linker comprises Structure 4:

A″-R3-B (Structure 4); and
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide;
A′ and A″ together comprise a first nucleophile and a first electrophile that react together to form A; and
B is a group comprising a second nucleophile or a second electrophile, wherein the second nucleophile is the same as the first nucleophile, and the second electrophile is the same as the first electrophile; and
the resulting compound is Structure 1: X—R1-R2-A-R3-B.

In an alternative embodiment of the method, the homo-bivalent covalent linker is reacted with a functionalized R2 end group to form an intermediate, and then the functionalized R2 end group of the intermediate is reacted with a functionalized substituent X that comprises a functional group reactive with the functionalized R2 end group to form R1; wherein: R1 is a group comprising phosphodiester, thiophosphodiester, sulfate, amide, triazole, heteroaryl, ester, ether, thioether, disulfide, thiopropionate, acetal, or glycol; and R2 is a spacer group. In an embodiment of this method, the homo-bivalent covalent linker comprises Structure 4:

A″-R3-B (Structure 4)

the functionalized R2 end group comprises Structure 5:

R1′-R2-A′ (Structure 5)

the functionalized substituent X comprises Structure 6:

X—R1″ (Structure 6); and
X is a substituent comprising a biological moiety other than a nucleic acid;
R1′ and R1″ are functional groups that react to form R1;
R1 is a group comprising phosphodiester, thiophosphodiester, sulfate, amide, triazole, heteroaryl, ester, ether, thioether, disulfide, thiopropionate, acetal, or glycol;
R2 is a spacer group;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide; and
A′ and A″ comprise a first nucleophile and a first electrophile that react to form A;
B is a group comprising a second nucleophile or a second electrophile, wherein the second nucleophile is the same as the first nucleophile, and the second electrophile is the same as the first electrophile; and
the resulting compound is Structure 1: X—R1-R2-A-R3-B.

In an embodiment of the method, the coupling of the homo-bivalent covalent linker to the substituent X is carried out in a dilute solution of the functionalized substituent X with a stoichiometric excess of the homo-bivalent covalent linker.

In an embodiment of the method, the coupling of the homo-bivalent covalent linker to the substituent X is carried out with a molar excess of the homo-bivalent covalent linker of at least about 5, 10, 15, 20, 25, 30, 35, 40, 45, 50, or 100.

In an embodiment of the method, the coupling of the homo-bivalent covalent linker to the substituent X is carried out with a molar excess of the homo-bivalent covalent linker of about 5, 10, 15, 20, 25, 30, 35, 40, 45, 50, or 100.

In an embodiment of the method, the coupling of the homo-bivalent covalent linker to the substituent X is carried out at a pH of below about 7, 6, 5, or 4.

In an embodiment of the method, the coupling of the homo-bivalent covalent linker to the substituent X is carried out at a pH of about 7, 6, 5, or 4.

In an embodiment of the method, the coupling of the homo-bivalent covalent linker to the substituent X is carried out in a solution comprising water and a water miscible organic co-solvent. In an embodiment, the water miscible organic co-solvent comprises DMF, NMP, DMSO, alcohol, or acetonitrile. In an embodiment, the water miscible organic co-solvent comprises about 10, 15, 20, 25, 30, 40, or 50% (v/v) of the solution.

In an embodiment of the method, the coupling of the homo-bivalent covalent linker to the substituent X is carried out in a solution comprising an anhydrous organic solvent. In a further embodiment, the anhydrous organic solvent comprises dichloromethane, DMF, DMSO, THF, dioxane, pyridine, alcohol, or acetonitrile.

In an embodiment of the method, the yield of the resulting mono-substituted covalent linker is at least 75, 80, 85, 90, 95, 96, 97, 98, 99, or 100%.

In an embodiment of the method, the purity of the resulting mono-substituted covalent linker is at least 75, 80, 85, 90, 95, 96, 97, 98, 99, or 100%.

In an embodiment, the disclosure provides a multi-conjugate comprising Structure 7:

X●Y (Structure 7)

wherein:
X is a first substituent comprising a biological moiety other than a nucleic acid;
Y is a second substituent that is the same as or different from X; and
● is a covalent linker joining X and Y, and comprising Structure 8:

—R1-R2-A-R3-A-R2-R1— (Structure 8)
wherein:
   each R1 is independently a group comprising phosphodiester, thiophosphodiester, sulfate, amide, triazole, heteroaryl, ester, ether, thioether, disulfide, thiopropionate, acetal, glycol, or is absent;
   each R2 is independently a spacer group, or is absent;
   each A is the same and is a group comprising the reaction product of a nucleophile and an electrophile; and
   R3 is a group comprising a C₂-C₁₀ alkyl, C₂-C₁₀ alkoxy, C₁-C₁₀ aryl, amide, C₂-C₁₀ alkyldithio, amide, ether, thioether, ester, oligonucleotide, oligopeptide, thiopropionate, or disulfide.

In an embodiment of the multi-conjugate of Structure 7, Y is different from X.

In an embodiment of the multi-conjugate of Structure 7, X is a peptide, protein, lipid, carbohydrate, carboxylic acid, vitamin, steroid, lignin, small molecule, organometallic compound, or a derivative of any of the foregoing.

In an embodiment of the multi-conjugate of Structure 7, Y is a nucleic acid, peptide, protein, lipid, carbohydrate, carboxylic acid, vitamin, steroid, lignin, small molecule, organometallic compound, or a derivative of any of the foregoing.

In an embodiment of the multi-conjugate of Structure 7, X is a peptide or a derivative of a peptide. In a further embodiment, X is the transduction domain of HIV-1TAT protein, or a derivative thereof. In an embodiment, the peptide comprises a Centryrin. In an embodiment, the peptide comprises a constrained peptide. In an embodiment, the peptide comprises a pHLIP peptide

In an embodiment of the multi-conjugate of Structure 7, X is an antibody or an antibody fragment, or a derivative thereof. In a further embodiment, X is an antibody single-chain variable fragment, or a derivative thereof.

In an embodiment of the multi-conjugate of Structure 7, X is a small molecule, or a derivative thereof. In a further embodiment, the small molecule is a therapeutic drug molecule. For example, in an embodiment, X is lenalidomide, or a derivative thereof.

In an embodiment of the multi-conjugate of Structure 7, X is an organometallic compound, or a derivative thereof. In a further embodiment, X is ferrocene, or a derivative thereof.

In an embodiment of the multi-conjugate of Structure 7, Y is nucleic acid, or a derivative thereof. In a further embodiment, Y is RNA or a derivative thereof. In a still further embodiment, Y is siRNA, saRNA, or miRNA, or derivatives thereof.

In an embodiment of the multi-conjugate of Structure 7, the covalent linker joining X and Y is cleavable under intracellular conditions.

In an embodiment, the multi-conjugate of Structure 7 is at least 75, 80, 85, 90, 95, 96, 97, 98, 99, or 100% pure.

In an embodiment, the disclosure provides a method for synthesizing a multi-conjugate of Structure 7:

X●Y (Structure 7)

wherein:
X is a first substituent comprising a biological moiety other than a nucleic acid;
Y is a second substituent that is the same as or different from X; and
● is a covalent linker joining X and Y;

the method comprising the steps of:
(a) reacting X—R4 with a homo-bivalent linker ∘ to produce a mono-substituted product X-∘, wherein R4 is a functional group capable of reacting with ∘ under conditions that produce the mono-substituted product X-∘ and substantially prevent dimerization of X; and
(b) reacting X-∘ with R5-Y, wherein R5 is a functional group capable of reacting with ∘, thereby forming X●Y.

In an embodiment, the disclosure provides a method for synthesizing a multi-conjugate of Structure 7:

X●Y (Structure 7)

wherein:
X is a first substituent comprising a biological moiety other than a nucleic acid;
Y is a second substituent that is the same or different from X; and
● is a covalent linker joining X and Y;

the method comprising the steps of:
(a) reacting R4-Y with a homo-bivalent linker ∘ to produce a mono-substituted product ∘-Y, wherein R4 is a functional group capable of reacting with ∘ under conditions that produce the mono-substituted product ∘-Y and substantially prevent dimerization of Y; and
(b) reacting ∘-Y with X—R5, wherein R5 is a functional group capable of reacting with ∘, thereby forming X●Y.

In an embodiment of the method for synthesizing a multi-conjugate of Structure 7, step (a) is carried out with a stoichiometric excess of the homo-bivalent linker ∘ relative to X—R4.

In an embodiment of the method for synthesizing a multi-conjugate of Structure 7, step (a) is carried out with a stoichiometric excess of the homo-bivalent linker ∘ relative to R4-Y.

In an embodiment of the method for synthesizing a multi-conjugate of Structure 7, step (a) is carried out with a molar excess of the homo-bivalent linker ∘ of about 5, 10, 15, 20, 25, 30, 35, 40, 45, 50, or 100.

In an embodiment of the method for synthesizing a multi-conjugate of Structure 7, step (a) is carried out in a solution comprising water and, optionally, a water miscible organic co-solvent. In a further embodiment, where a water miscible organic co-solvent is used, the water miscible organic co-solvent comprises DMF, DMSO, THF, dioxane, pyridine, alcohol, or acetonitrile. In a further embodiment, the water miscible organic co-solvent comprises about 10, 15, 20, 25, 30, 40, or 50% (v/v) of the solution

Alcohols for use in the above-described methods of synthesis include but are not limited to C₁-C₁₀ alcohol, C₁-C₇ alcohol, and C₁-C₅ alcohol, in each case optionally substituted with a water miscibility enhancing group such as amino, tertiary amino, or sulfate.

In an embodiment of the method for synthesizing a multi-conjugate of Structure 7, step (a) is carried out at a pH of about 7, 6, 5, or 4.

In an embodiment of the method for synthesizing a multi-conjugate of Structure 7, step (a) is carried out in a solution comprising an anhydrous organic solvent. In a further embodiment, the anhydrous organic solvent comprises dichloromethane, DMF, DMSO, THF, dioxane, pyridine, alcohol, or acetonitrile.

In an embodiment of the method for synthesizing a multi-conjugate of Structure 7, X is a peptide, protein, lipid, carbohydrate, carboxylic acid, vitamin, steroid, lignin, small molecule, organometallic compound, or a derivative of any of the foregoing.

In an embodiment of the method for synthesizing a multi-conjugate of Structure 7, Y is a nucleic acid, peptide, protein, lipid, carbohydrate, carboxylic acid, vitamin, steroid, lignin, small molecule, or a derivative of any of the foregoing.

In an embodiment of the method for synthesizing a multi-conjugate of Structure 7, X is a peptide, or a derivative of a peptide. In a further embodiment, X is the transduction domain of HIV-1TAT protein, or a derivative thereof. In an embodiment, the peptide comprises a Centryrin. In an embodiment, the peptide comprises a constrained peptide. In an embodiment, the peptide comprises a pHLIP peptide

In an embodiment of the method for synthesizing a multi-conjugate of Structure 7, X is an antibody, or a fragment thereof. In a further embodiment, X is a single-chain antibody variable fragment, or a derivative thereof.

In an embodiment of the method for synthesizing a multi-conjugate of Structure 7, X is a small molecule, or a derivative thereof. In a further embodiment, the small molecule is a therapeutic drug molecule. For example, in an embodiment, X is lenalidomide, or a derivative thereof.

In an embodiment of the method for synthesizing a multi-conjugate of Structure 7, X is an organometallic compound, or a derivative thereof. In a further embodiment, X is ferrocene, or a derivative thereof.

In an embodiment of the method for synthesizing a multi-conjugate of Structure 7, Y is a nucleic acid, or a derivative thereof. In a further embodiment, Y is RNA or a derivative thereof. In a still further embodiment, Y is siRNA, saRNA, or miRNA.

In an embodiment of the method for synthesizing a multi-conjugate of Structure 7, the covalent linker ● is cleavable under intracellular conditions.

In an embodiment of the method for synthesizing a multi-conjugate of Structure 7, the yield of X●Y is at least 75, 80, 85, 90, 95, 96, 97, 98, 99 or 100%.

In an embodiment of the method for synthesizing a compound of Structure 7, the purity of X●Y is at least 75, 80, 85, 90, 95, 96, 97, 98, 99 or 100%.

In an embodiment, the disclosure provides a multi-conjugate comprising substituents X, Y and Z, wherein each of the substituents, independently, is a biological moiety and is joined to another substituents by a covalent linker ●; wherein the multi-conjugate comprises Structure 9:

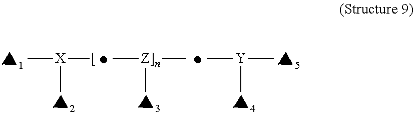

wherein:
each of ▴₁, ▴₂, ▴₃, ▴₄ and ▴₅ is, independently, absent or comprises a biological moiety covalently linked to its respective substituent;
n is an integer that is greater than or equal to zero; and
at least one of the substituents present in Structure 9 is not a nucleic acid.

In an embodiment of the multi-conjugate of Structure 9, n is 1, 2, 3, 4, 5, 6, 7, 8, 9, or 10.

In an embodiment of the multi-conjugate of Structure 9, at least one covalent linker ● is a homo-bivalent covalent linker.

In an embodiment of the multi-conjugate of Structure 9, X is not a nucleic acid.

In an embodiment of the multi-conjugate of Structure 9, Y is not a nucleic acid.

In an embodiment of the multi-conjugate of Structure 9, at least ▴ is present. In a further embodiment, the at least one ▴ that is present is a targeting ligand. In an embodiment, at least two ▴ are present and each of the two ▴ that are present are a targeting ligand. In an embodiment, at least two ▴ are present and each of the two ▴ that are present are the same targeting ligand. In an embodiment, ▴1 and ▴5 are present and are the same targeting ligand.

In an embodiment of the multi-conjugate of Structure 9, at least one covalent linker ● is a sulfur-containing covalent linker; and at least one of ▴₁, ▴₂, ▴₃, ▴₄ and ▴₅ comprises a sulfur-containing end group Q. In a further embodiment, the sulfur-containing end group Q comprises a protected thiol group that is deprotectable under a deprotection condition; and the sulfur-containing covalent linker ● is stable under the deprotection condition. In another embodiment, the sulfur-containing covalent linker ● comprises a cleavable group that is cleavable under a cleavage condition that is not the deprotection condition. In a further embodiment, the sulfur-containing end group Q comprises a protected thiol group of the formula S-PG.

In an embodiment, the disclosure provides a method for synthesizing a multi-conjugate comprising Structure 10:

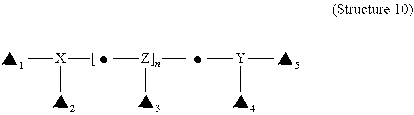

the method comprising:
reacting a compound of Structure 10a with a homo-bivalent linker to form a compound of Structure 10b under conditions that produce the mono-substituted product (Structure 10b) and substantially prevent dimerization of Structure 10a;
reacting the compound of Structure 10b with a compound of Structure 10c to form a compound of Structure 10d;
deprotecting the compound of Structure 10d to form a compound of Structure 10e; and
reacting the compound of Structure 10e with a compound of Structure 10f to form Structure 10; as follows:

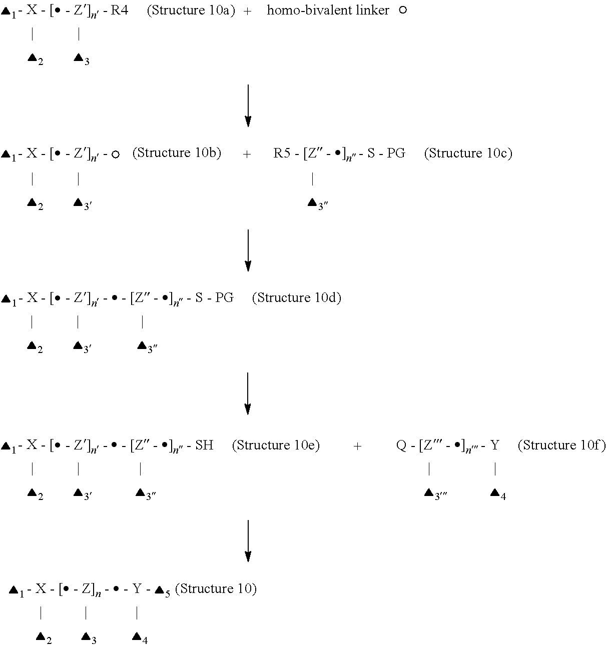

wherein:
● is a covalent linker;
∘ is a homo-bivalent linker;
R4 is a functional group selected to react with the homo-bivalent linker ∘ under conditions that produce the mono-substituted product of Structure 10b and substantially prevent dimerization of Structure 10a;
R5 is a functional group selected to react with the homo-bivalent linker ∘;
S-PG is a protected thiol group comprising a sulfur-containing group that is different from any sulfur-containing group residing within any of the covalent linkers ● within Structures 10b, 10c and 10d;
Q is a reactive group selected to react with the —SH group of Structure 10e to form a covalent linker ●;
X, Y, and Z are substituents of the multi-conjugate, and each is a biological moiety;
Z′, Z″, and Z′″ are substituents of the multi-conjugate, and each is a biological moiety;
each of ▴₁, ▴₂, ▴₃, ▴₄ and ▴₅ is, independently, absent or comprises a biological moiety covalently linked to its respective substituent;
each of ▴_{3′}, ▴_{3″}, and ▴_{3′″} is, independently, absent or comprises a biological moiety covalently linked to its respective substituent;
n is an integer that is greater than or equal to 1, and optionally n is 1, 2, 3, 4, 5, 6, 7, 8, 9, or 10; and
n′, n″, and n′″ are each an integer greater than or equal to zero, with the proviso that the sum of n′+n″+n′″ is n.

In an embodiment of a method for synthesizing a multi-conjugate comprising Structure 10, at least one of X, Y, and Z that is present in the multi-conjugate is not a nucleic acid.

The disclosure provides compositions comprising the disclosed multi-conjugates and a pharmaceutically acceptable excipient.

The disclosure provides the disclosed multi-conjugates for use in the manufacture of a medicament.

The disclosure provides methods for treating a subject in need of medical treatment or prophylaxis comprising the administration of an effective amount of any of the various multi-conjugates disclosed herein.

The disclosure provides methods for modulating an activity of one or more target genes in a cell, the method comprising introducing to the cell any of the various multi-conjugates disclosed herein and maintaining the cell under conditions in which the multi-conjugate enters the cell, and the activity of the target gene or genes is modulated.

The disclosure provides methods for observing an activity of a multi-conjugate in a cell, the method comprising introducing to the cell any of the various multi-conjugates disclosed herein and maintaining the cell under conditions in which the multi-conjugate enters the cell, and the activity of the multi-conjugate is observed.

These and other embodiments are described in greater detail below.

While the disclosure comprises embodiments in many different forms, there will herein be described in detail several specific embodiments with the understanding that the present disclosure is to be considered as an exemplification of the principles of the technology and is not intended to limit the disclosure to the embodiments illustrated.

### DETAILED DESCRIPTION

The disclosures of any patents, patent applications, and publications referred to herein are hereby incorporated by reference in their entireties into this application in order to more fully describe the state of the art known to those skilled therein as of the date of the disclosure described and claimed herein

As used herein, the term “about” is used in accordance with its plain and ordinary meaning of approximately. For example, “about X” encompasses approximately the value X as stated, including similar amounts that are within the measurement error for the value of X, or amounts that are approximately the same as X and have essentially the same properties as X.

As used herein, the term “isolated” includes compounds that are separated from other, unwanted substances. The isolated compound can be synthesized in a substantially pure state or separated from the other components of a crude reaction mixture, except that some amount of impurities, including residual amounts of other components of the crude reaction mixture, may remain. Similarly, “pure” or “substantially pure” means sufficiently free from impurities to permit its intended use (e.g., in a pharmaceutical formulation or as a material for a subsequent chemical reaction). X % pure means that the compound is X % of the overall composition by relevant measure, which can be for example by analytical methods such as HPLC.

Biological Moieties

As used herein, the term “biological moiety” has its ordinary meaning as understood by those skilled in the art. It refers to chemical entities that are biologically active or inert when delivered into a cell or organism.

In many instances, a biological moiety will produce a biological effect or activity within the cell or organism to which it is delivered; and oftentimes the biological effect or activity is detectable or measurable. In other instances, a biological moiety may be selected to augment or enhance the biological effect or activity of another biological moiety with which it is delivered. In still other instances, a biological moiety may be selected for use in a method for synthesizing a synthetic intermediate or multi-conjugate (as described below).

Examples of biological moieties include but are not limited to nucleic acids, amino acids, peptides, proteins, lipids, carbohydrates, carboxylic acids, vitamins, steroids, lignins, small molecules, organometallic compounds, or derivatives of any of the foregoing.

In some aspects of the disclosure, the biological moiety is a moiety other than a nucleic acid (a “non-nucleic acid biological moiety”). Non-nucleic acid biological moieties include but are not limited to amino acids, peptides, proteins, lipids, carbohydrates, carboxylic acids, vitamins, steroids, lignins, small molecules (e.g., a small molecule therapeutic or drug molecule), organometallic compounds, or derivatives of any of the foregoing

In some aspects of the disclosure, a biological moiety may comprise a cell- or tissue-targeting moiety, such as but not limited to a ligand specific for a given cell-surface receptor. Examples of cell- or tissue-targeting moieties include but are not limited to lipophilic moieties, such as phospholipids; aptamers (of DNA or RNA, or derivatives thereof); peptides and proteins, such as antigen-binding peptides or proteins; small molecules; vitamins, such as tocopherol and folate; other folate receptor-binding ligands; carbohydrates, such as N-Acetylgalactosamine (GalNAc) and mannose; other mannose-receptor binding ligands; cholesterols; carboxylic acids, such as 2-[3-(1,3-dicarboxypropyl)-ureido]pentanedioic acid (DUPA); and derivatives of benzamide, such as anisamide.

A cell- or tissue-targeting lipophilic moiety may comprise a cationic group. In some aspects of the present disclosure, the lipophilic moiety comprises a cholesterol, vitamin E, vitamin K, vitamin A, folic acid, or a cationic dye (e.g., Cy3). Other lipophilic moieties include but are not limited to cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

Examples of antigen-binding proteins include but are not limited to monoclonal antibodies, single chain variable fragments (ScFv) or VHH antigen-binding proteins.

Examples of GalNAc-based biological moieties include but are not limited to mono-antennary GalNAc, di-antennary GalNAc, and tri-antennary GalNAc.

Other biological moieties, some or all of which may have cell- or tissue-targeting properties, include but are not limited to fatty acids, such as cholesterol; Lithocholic acid (LCA); Eicosapentaenoic acid (EPA); Docosahexaenoic acid (DHA); Docosanoic acid (DCA); steroids; secosteroids; lipids; gangliosides; nucleoside analogs; endocannabinoids; vitamins such as choline, vitamin A, vitamin E, retinoic acid and tocopheryl; and derivatives of any of the foregoing.

Other peptide-based biological moieties, some or all of which may have cell- or tissue-targeting properties, include but are not limited to: APRPG, cNGR (CNGRCVSGCAGRC), F3 (KDEPQRRSARLSAKPAPPKPEPKPKKAPAKK), CGKRK, and/or iRGD (CRGDKGPDC). Other peptides within the scope of this disclosure include:
(a) Centyrins, which are small single-domain proteins derived from the human Tenascin C protein and engineered to bind to targets with high selectivity and affinity (see Klein et al., Centyrin ligands for extrahepatic delivery of siRNA, MOLECULAR THERAPY Vol. 29 No 6 (June 2021) and Mahalingam et al., Evaluation of a Centyrin-Based Near-Infrared Probe for Fluorescence-Guided Surgery of Epidermal Growth Factor Receptor Positive Tumors, BIOCONJUGATE CHEM. 2017, 28, 11, 2865-2873 (Sep. 25, 2017) (each of which is herein incorporated by reference in its entirety); (b) constrained peptides, which include macrocyclic and stapled peptides as described in Cary et al., Constrained Peptides in Drug Discovery and Development, J. SYNTH. ORG. CHEM., JPN, xxx, Vol. 75 No. 11 (2017) (herein incorporated by reference in its entirety); and
(c) pH (low) insertion peptides (pHLIPs), which target acidity at the surfaces of cells as described in Wyatt et al., Peptides of pHLIP family for targeted intracellular and extracellular delivery of cargo molecules to tumors, PNAS, vol. 115, no. 12, E2811-E2818 (2018) (herein incorporated by reference in its entirety).

In various aspects, the disclosure provides for the use and incorporation of nuclear localization signals or sequences (NLS) to facilitate importation of a material to which it is linked or incorporated to the cellular nucleus. The NLS is typically an amino acid sequence, examples of which are known by those working in the field of drug delivery.

In some aspects of the disclosure, a biological moiety may comprise an endosomal escape moiety (EEM), selected to assist or enable other biologically active moieties with which it is delivered to disrupt the endosomal membrane or otherwise to escape the endosome or other organelle within which the biological moiety is internalized (such as by endocytosis) upon intracellular delivery. Endosomal escape moieties are oftentimes lipid-based or amino acid-based, but may comprise other chemical entities that disrupt an endosome to release its cargo. Examples of EEMs include but are not limited to chloroquine, peptides and proteins with motifs containing hydrophobic amino acid R groups, and influenza virus hemagglutinin (HA2). Further EEMs are described in Lonn et al., Scientific Reports, 6: 32301, 2016.

Other examples of cell- and tissue-targeting moieties and EEMs are provided below in the section entitled Delivery Constructs and Formulations.

In some aspects of the disclosure, a biological moiety may comprise an immunomodulator, such as an immunosuppressive agent or an immunostimulatory agent.

Additional biological moieties within the scope of the disclosure are any of the known gene editing materials, including for example, materials such as oligonucleotides, polypeptides and proteins involved in CRISPR/Cas systems, TALES, TALENs, and zinc finger nucleases (ZFNs).

Further, biological moieties within the scope of the disclosure may comprise a detectable label. As used herein, “detectable label” has its ordinary meaning as understood by those skilled in the art. It refers to a chemical group that is a substituent of a multi-conjugate and detectable by an imaging technique, such as fluorescence spectroscopy. For example, the detectable label may be a dye that comprises a fluorophore, which, after absorption of energy, emits radiation at a defined wavelength. Many suitable fluorescent labels or dyes are known. For example, Welch et al. (Chem. Eur. J. 5(3):951-960, 1999) discloses dansyl-functionalised fluorescent moieties and Zhu et al. (Cytometry 28:206-211, 1997) describes the use of the fluorescent labels Cy3 and Cy5. Other labels are described in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321:674, 1986). Examples of commercially available fluorescent labels include, but are not limited to, fluorescein, rhodamine (such as TMR, texas red or Rox), alexa, bodipy, acridine, coumarin, pyrene, benzanthracene and cyanine (such as Cy2 or Cy4). Other forms of detectable labels include microparticles, including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci USA 97(17):9461-9466, 2000), and tags detectable by mass spectrometry. The detectable label may be a multi-component label that is dependent on an interaction with another compound for detection, such as the biotin-streptavidin system.

In many aspects of the disclosure, a biological moiety may comprise an oligonucleotide, including but not limited to RNA, DNA, a combination thereof, or comprising an artificial or non-natural nucleic acid analog. In various embodiments, the oligonucleotide is single-stranded. In various embodiments, the oligonucleotide is double-stranded (e.g., antiparallel double-stranded).

In various embodiments, the oligonucleotide is RNA, for example an antisense RNA (aRNA), CRISPR RNA (crRNA), long noncoding RNA (lncRNA), microRNA (miRNA), piwi-interacting RNA (piRNA), small interfering RNA (siRNA), messenger RNA (mRNA), short hairpin RNA (shRNA), small activating (saRNA), or ribozyme.

In various embodiments, the oligonucleotide is a DNA or RNA aptamer.

In some embodiments, the oligonucleotide is a CRISPR guide RNA, or other RNA associated with or essential to forming a ribonucleocomplex (RNP) with a Cas nuclease in vivo, in vitro, or ex vivo, or associated with or essential to performing a genomic editing or engineering function with a Cas nuclease, including for example wild-type Cas nuclease, or any of the known modifications of wild-type Cas, such as nickases and dead Cas (dCas). CRISPR-Cas systems are described, for example, in U.S. Pat. No. 8,771,945; Jinek et al., Science, 337(6096): 816-821 (2012), and International Patent Application Publication No. WO 2013/176772.

In various embodiments, the oligonucleotide is 15-30, 17-27, 19-26, 20-25, 40-50, 40-150, 100-300, 1000-2000, or up to 10000 nucleotides in length.

In various embodiments, the oligonucleotide is double-stranded and complementary. Complementarity can be 100% complementary, or less than 100% complementary where the oligonucleotide nevertheless hybridizes and remains double-stranded under relevant conditions (e.g., physiologically relevant conditions). For example, a double-stranded oligonucleotide can be at least about 80%, 85%, 90%, or 95% complementary. In some embodiments, the double-stranded oligonucleotide is blunt-ended (a symmetric oligonucleotide). In some embodiments, the double-stranded oligonucleotide has a terminal overhang on one strand (e.g., 2-5 overhanging nucleotides) or a terminal overhang on each of its strands (in each case, an asymmetric oligonucleotide).

In some embodiments, the oligonucleotide is DNA, for example an antisense DNA (aDNA) (e.g., antagomir) or antisense gapmer. Examples of aDNA, including gapmers and multimers, are described for example in Subramanian et al., Nucleic Acids Res, 43(19): 9123-9132 (2015) and International Patent Application Publication No. WO 2013/040429. Examples of antagomirs are described for example, in U.S. Pat. No. 7,232,806.

In various embodiments, the oligonucleotide according to the disclosure further comprises a chemical modification. The chemical modification can comprise a modified nucleoside, modified backbone, modified sugar, and/or modified terminus.

Modifications may include phosphorus-containing linkages, which include but are not limited to phosphorothioates, enantiomerically enriched phosphorothioates, phosphorodithioates, phosphotriesters, aminoalkylphosphotriesters, methyl and other alkyl phosphonates comprising 3′alkylene phosphonates and enantiomerically enriched phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

In various embodiments, the oligonucleotides may comprise one or more phosphorothioate groups. The oligonucleotides may comprise one to three phosphorothioate groups at the 5′ end. The oligonucleotides may comprise one to three phosphorothioate groups at the 3′ end. The oligonucleotides may comprise one to three phosphorothioate groups at the 5′ end and the 3′ end. In various embodiments, each oligonucleotide contained in the multi-conjugate may comprise 1-10 total phosphorothioate groups. In certain embodiments, each oligonucleotide may comprise fewer than 10, fewer than 9, fewer than 8, fewer than 7, fewer than 6, fewer than 5, fewer than 4, or fewer than 3 total phosphorothioate groups. In certain embodiments, the oligonucleotides contained in the multi-conjugate may possess increased in vivo activity with fewer phosphorothioate groups relative to the same oligonucleotides in monomeric form with more phosphorothioate groups.

The oligonucleotides may be modified using various strategies known in the art to produce a variety of effects, including, e.g., improved potency and stability in vitro and in vivo. Among these strategies are: artificial nucleic acids, e.g., 2′-O-methyl-substituted RNA; 2′-fluro-2′deoxy RNA, peptide nucleic acid (PNA); morpholinos; locked nucleic acid (LNA); Unlocked nucleic acids (UNA); bridged nucleic acid (BNA); glycol nucleic acid (GNA); and threose nucleic acid (TNA); or more generally, nucleic acid analogs, e.g., bicyclic and tricyclic nucleoside analogs, which are structurally similar to naturally occurring RNA and DNA but have alterations in one or more of the phosphate backbone, sugar, or nucleobase portions of the naturally-occurring molecule. Typically, analogue nucleobases confer, among other things, different base pairing and base stacking properties. Examples include universal bases, which can pair with all four canon bases. Examples of phosphate-sugar backbone analogues include, but are not limited to, PNA. Morpholino-based oligomeric compounds are described in Braasch et al., Biochemistry, 41(14): 4503-4510 (2002) and U.S. Pat. Nos. 5,539,082; 5,714,331; 5,719,262; and 5,034,506.

In the manufacturing methods described herein, some of the oligonucleotides are modified at a terminal end by substitution with a chemical functional group. The substitution can be performed at the 3′ or 5′ end of the oligonucleotide, and may be performed at the 3′ ends of both the sense and antisense strands of the monomer, but is not always limited thereto. The chemical functional groups may include, e.g., a sulfhydryl group (—SH), a carboxyl group (—COOH), an amine group (—NH2), a hydroxy group (—OH), a formyl group (—CHO), a carbonyl group (—CO—), an ether group (—O—), an ester group (—COO—), a nitro group (—NO₂), an azide group (—N₃), or a sulfonic acid group (—SO₃H).

The oligonucleotides may be modified to, additionally or alternatively, include nucleobase (referred to in the art simply as “base”) modifications or substitutions. Modified nucleobases include nucleobases found only infrequently or transiently in natural nucleic acids, e.g., hypoxanthine, 6-methyladenine, 5-Me pyrimidines, 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77 (1980); Gebeyehu et al., Nucl. Acids Res, 15: 4513 (1997). A “universal” base known in the art, e.g., inosine or pseudouridine, can also be included. 5-Me-C substitutions can increase nucleic acid duplex stability by 0.6-1.2° C. (Sanghvi, Y. S., in Crooke, S. T. and Lebleu, B., eds., Antisense Research and Applications, CRC Press, Boca Raton, pp 276-278 (1993) and are aspects of base substitutions. Modified nucleobases can include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo, such as 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine. Hydroxy group (—OH) at a terminus of the nucleic acid can be substituted with a functional group such as sulfhydryl group (—SH), carboxyl group (—COOH) or amine group (—NH₂). The substitution can be performed at the 3′ end or the 5′ end.

Further examples of biological moieties are described in WO2016/205410, WO2018/145086, and WO 2020/180897, each of which is incorporated herein by reference.

Multi-Conjugates

As used herein, the term “multi-conjugate” has its ordinary meaning as understood by those skilled in the art. It refers to compounds that comprise two or more substituents joined to one another by a covalent linker, wherein each of the substituents are, independently, a biological moiety.

Examples of multi-conjugates include but are not limited to small molecules (e.g., biotin) conjugated proteins, protein-protein conjugates (e.g., an antibody coupled to an enzyme), conjugates commonly known as antibody drug conjugates (ADCs) (e.g., a monoclonal antibody conjugated to a cytotoxic small molecule), radio-immunoconjugates (e.g., a monoclonal antibody conjugated to a chelating agent), vaccines (e.g., haptens conjugated to carrier proteins), antibodies conjugated to nanoparticles and non-cytotoxic drugs (e.g., peptides), biomolecules conjugated to elements or derivatives thereof (e.g., TGF-β conjugated to iron oxide nanoparticles), and others as described herein.

Covalent Linkers

In various aspects and embodiments of the disclosure, multiple biological moieties are linked covalently to form multi-conjugates. Throughout the disclosure and the claims, a covalent linker in all of its representations, including but not limited to “●”, may optionally include a spacer group □ on one or both sides of the covalent linkage, as in □-●, □-●-□, or ●-□, unless expressly stated otherwise.

Covalent linkers may be cleavable or noncleavable. Cleavable linkers may be selected or designed to maintain stability upon administration, while cleaving upon delivery, or under intracellular conditions, to facilitate functional delivery of the biological moieties. In addition to the examples of covalent linkers provided herein, those having ordinary skill will recognize that a wide variety of covalent linkers, including their composition, synthesis, and use are known in the art, and may be adapted for use consistent with this disclosure.

Nucleotide linkers are one example of a class of covalent linkers, including for example, nucleic acid sequences such as Uridine-Uridine-Uridine (UUU), the endonuclease cleavable linker dCdA, and dTdTdTdT. Nucleotide linkers contain one or more nucleotides selected such that the sequence does not carry out any other designated function. In various aspects of the disclosure, a covalent linker can comprise a nucleotide linker of 2-6 nucleotides in length.

In various aspects of the disclosure, a covalent linker comprises the reaction product of nucleophilic and electrophilic groups. For example, a covalent linker can comprise the reaction product of a thiol and maleimide, a thiol and vinylsulfone, a thiol and pyridyldisulfide, a thiol and iodoacetamide, a thiol and acrylate, an azide and alkyne, or an amine and carboxyl group. As described herein, one of these groups is connected, e.g., to a substituent of the multi-conjugate (e.g., a thiol (—SH) functionalization on the substituent) and the other group presents on a second molecule (e.g., a linking agent) that ultimately links two oligonucleotides (e.g., maleimide in DTME).

Covalent linkers comprising the reaction product of a thiol and maleimide, include but are not limited to DTME (dithiobismaleimidoethane), BM(PEG)2 (1,8-bis(maleimido)diethylene glycol), BM(PEG)3 (1,11-bismaleimido-triethyleneglycol), BMOE (bismaleimidoethane), BMH (bismaleimidohexane), or BMB (1,4-bismaleimidobutane). DTME is advantageous in that it contains an internal disulfide which is cleavable intracellularly, in the reductant environment of the cytosol.

In various examples and aspects of the disclosure, the covalent linker is bivalent, meaning that it has two sites for reaction with a biological moiety. A “homo-bivalent” covalent linker refers to a linker in which the two reactive sites are the same (e.g., the two maleimides in dithio-bis-maleimidoethane [DTME]). Those having ordinary skill will recognize that a variety of homo-bivalent covalent linkers may be adapted for use consistent with this disclosure.

Other cleavable homo-bivalent covalent linkers include but are not limited to compounds comprising Structure 11:

X—R-[p(Np)ₐ-Rp(Np)_{b}-Rp(Np)_{c}-Rp(Np)_{d}]-R—X (Structure 11)

wherein each X is independently a functional group; each R is independently a spacer group or is absent; each p is independently a derivative of phosphoric acid or is absent, with the proviso that Structure 11 must contain at least one p; each N is independently a nucleoside or is absent; and each of a, b, c, and d are independently an integer in the range of 0 to 4 inclusive, with the proviso that the sum of a+b+c+d must be greater than or equal to 1. In an embodiment, Structure 11 must contain at least one nucleoside N. In an embodiment, the sum of a+b+c+d must be greater than or equal to 2.

Other cleavable homo-bivalent covalent linkers include but are not limited to compounds comprising identical functional groups at either end, wherein said functional groups are joined by a covalent linker comprising at least one amide bond. In an embodiment, the compound comprises Structure 12:

(X—)-□-(—X) Structure 12

wherein,
(X—) and (—X) are representations of a functional group, X, linked to a spacer group, —; and
□ is a region of the compound comprising at least one amide bond.

For example, an embodiment provides a homo-bivalent linker compound comprising identical functional groups at either end, wherein said functional groups are joined by a covalent linker comprising at least one amide bond, wherein the compound comprises Structure 12a:

(X—)[R-Aₐ-B_{b}-C_{c}-Dₐ-R′](—X) Structure 12a

wherein:
(X—) and (—X) are representations of a functional group, X, linked to a spacer group, —;
R is H, or is absent;
R′ is OH, or is absent;
each of a, b, c, and d is independently 0 or 1, with the proviso that the sum of a+b+c+d is greater than or equal to 2; and
each of A, B, C and D independently comprises Structure 12b:

[N▴ [symbol] -(CH [symbol] )_{w}-(CH [symbol] )ₓ-(CH [symbol] )_{y}-(CH [symbol] )_{z}-CO-▾] (Structure 12b)
wherein:
   each of w, x, y, and z are independently 0 or 1, with the proviso that the sum of w+x+y+z is greater than or equal to 1;
   each ▴ is independently H, H₂, alkyl, alkoxy, alkyl carboxy, alkyl carboxamide, alkyl amino, alkyl sulfate, aryl, aryl carboxy, aryl carboxamide, aryl amino, aryl sulfate or is absent;
   each of [symbol] , [symbol] , [symbol] , [symbol] , and [symbol] is independently present or absent, and if present designates a terminus of a cyclic group as follows:
      [symbol] designates the N in N▴ [symbol] as a terminus;
      [symbol] designates the C in (CH [symbol] )_{w} as a terminus;
      [symbol] designates the C in (CH [symbol] )ₓ as a terminus;
      [symbol] designates the C in (CH [symbol] )_{y} as a terminus; and
      [symbol] designates the C in (CH [symbol] )_{z} as a terminus;
      with the proviso that each Structure 12b independently contains zero, one or two cyclic groups, the termini of each cyclic group being selected from:
      [symbol] as a first terminus and [symbol] , [symbol] , [symbol] , or [symbol] as a second terminus;
      [symbol] as a first terminus and [symbol] , [symbol] , or [symbol] as a second terminus;
      [symbol] as a first terminus and [symbol] or [symbol] as a second terminus; or
      [symbol] as a first terminus and [symbol] as a second terminus;
      with the further proviso that:
      if [symbol] is present, then [symbol] is absent;
      if [symbol] is present, then [symbol] is absent;
      if [symbol] is present, then [symbol] is absent;
      if [symbol] is present, then [symbol] is absent;
   each cyclic group that is present in Structure 12b further comprises, in addition to its respective termini, a middle section between the termini, Y; and each Y is independently alkyl, alkoxy, alkyl carboxy, alkyl carboxamide, alkyl amino, or alkyl sulfate;
   each of [symbol] , [symbol] , [symbol] , and [symbol] are independently present or absent, and if present are H, OH, alkyl, alkyl carboxy, alkyl carboxamide, alkyl amino, alkoxy, thioalkyl, alkylthioalkyl, aryl, or heteroaryl;
   each of [symbol] , [symbol] , [symbol] , and [symbol] are, where present, optionally bonded to (—X), which is a representation of a functional group, X, linked to a spacer group, —
   each ▾ is independently OH, alkyl, alkoxy, alkyl carboxy, alkyl carboxamide, alkyl amino, alkyl sulfate, aryl, aryl carboxy, aryl carboxamide, aryl amino, aryl sulfate, or is absent; and
with the proviso that the resulting Structure 12a contains only two functional groups X linked via spacer groups — to the compound, in keeping with the compound being a homo-bivalent linker compound.

In various aspects of the disclosure, a covalent linker can comprise a disulfide bond or a compound of Structure 13:

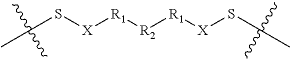

wherein S is attached by a covalent bond or by a linker to a substituent; each R1 is independently a C₂-C₁₀ alkyl, C₂-C₁₀ alkoxy, C₁-C₁₀ aryl group; R2 is a thiopropionate or disulfide group; and each X is independently selected from:

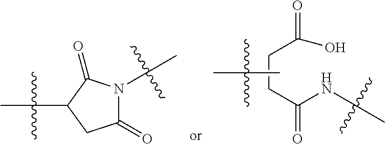

the latter structure representing two “ring-opened” positional isomers that are derivatives of succinamic acid.

In certain embodiments, the compound of Structure 13 is

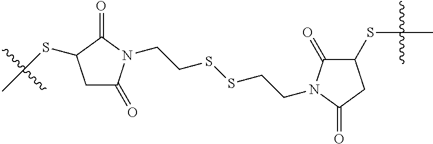

wherein S is attached by a covalent bond or by a linker to a substituent.

In certain embodiments, the compound of Structure 13 is

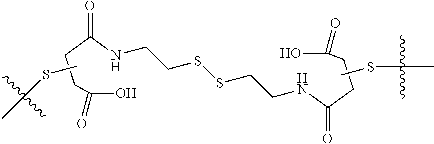

wherein both rings are opened (as shown, representing the various positional isomers), wherein each S is bonded to either of the alpha carbon atoms on the respective ring opened structures and wherein S is attached by a covalent bond or by a linker to a substituent.

In certain embodiments, the compound of Structure 13 is

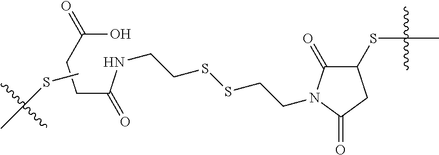

wherein one ring is opened (as shown, representing both positional isomers), wherein an S is bonded to either of the alpha carbon atoms on the respective ring opened structure, and wherein S is attached by a covalent bond or by a linker to a substituent.

In various embodiments, the covalent linker of Structure 13 is formed from a covalent linking precursor of Structure 14:

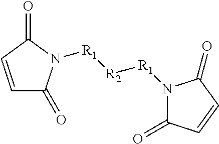

wherein each R1 is independently a C₂-C₁₀ alkyl, C₂-C₁₀ alkoxy, or C₁-C₁₀ aryl group; and R2 is a thiopropionate or disulfide group.

In various aspects of the disclosure, two or more linkers within a multi-conjugate can comprise orthogonal types of linkages, including, e.g., bio-cleavable linkages. For example, the two orthogonal bio-cleavable linkages can comprise a nucleic acid or oligopeptide linker on the one hand, and a linker comprising a reaction product of a nucleophile and an electrophile (e.g., a thiol and maleimide) on the other.

In various aspects of the disclosure, the covalent linker may comprise a non-ionic hydrophilic polymer such as polyethyleneglycol (PEG), polyvinylpyrolidone and polyoxazoline, or a hydrophobic polymer such as PLGA and PLA.

Polymer linking agents used as a mediator for a covalent bond include but are not limited to non-ionic hydrophilic polymers including polyethylene glycol (PEG), Pluronic, polyvinylpyrolidone, polyoxazoline, or copolymers thereof; or one or more biocleavable polyester polymers including poly-L-lactic acid, poly-D-lactic acid, poly-D,L-lactic acid, poly-glycolic acid, poly-D-lactic-co-glycolic acid, poly-L-lactic-co-glycolic acid, poly-D,L-lactic-co-glycolic acid, polycaprolactone, polyvalerolactone, polyhydroxybutyrate, polyhydroxyvalerate, or copolymers thereof.

The linking agent may have a molecular weight of about 100 to 10,000 Daltons. Examples of such linking agents include dithio-bis-maleimidoethane (DTME), 1,8-bis-maleimidodiethyleneglycol (BM(PEG)2), tris-(2-maleimidoethyl)-amine (TMEA), tri-succinimidyl aminotriacetate (TSAT), 3-arm-poly(ethylene glycol) (3-arm PEG), maleimide, N-hydroxysuccinimide (NHS), vinylsulfone, iodoacetyl, nitrophenyl azide, isocyanate, pyridyldisulfide, hydrazide, and hydroxyphenyl azide

Linking agents comprising cleavable bonds or noncleavable bonds may be used herein, and indeed, in some instances, may be used together in the same multi-conjugate. Linking agents comprising noncleavable bonds include but are not limited to those comprising an amide bond or a urethane bond. Linking agents comprising cleavable bonds include but are not limited to those comprising an acid cleavable bond (e.g., a covalent bond of ester, hydrazone, or acetal), a reductant cleavable bond (e.g., a disulfide bond), a bio-cleavable bond, or an enzyme cleavable bond (e.g., nucleic acid-based linkers or oligopeptide-based linkers). In some instances, the cleavable covalent linker is cleavable under intracellular conditions. Additionally, any linking agent available for drug conjugation can be used in the foregoing aspects of the invention without limitation.

Further, combinations of functional groups and linking agents may include: (a) where the functional groups are amino or thiol, the linking agent may be Succinimidyl 3-(2-pyridyldithio)propionate, or Succinimydyl 6-([3(2-pyridyldithio)propioamido]hexanoate; (b) where the functional group is amino, the linking agent may be 3,3′dithiodipropionic acid di-(N-succinimidyl ester), Dithio-bis(ethyl 1H-imidazole-1-carboxylate), or Dithio-bis(ethyl 1H-imidazole-1-carboxylate); (c) where the functional groups are amino or alkyne, the linking agent may be Sulfo-N-succinimidyl3-[[2-(p-azidosalicylamido)ethyl]-1,3′-dithio]propionate; and (d) where the functional group is thiol, the linking agent may be dithio-bis-maleimidoethane (DTME), 1,8-Bis-maleimidodiethyleneglycol (BM(PEG)2), or dithiobis(sulfosuccinimidyl propionate) (DTSSP).

In various methods for preparing and synthesizing the synthetic intermediates and multi-conjugates provided herein, there may be a step involving the activation of a functional group. Compounds that can be used in the activation of functional groups include but are not limited to 1-ethyl-3,3-dimethylaminopropyl carbodiimide, imidazole, N-hydroxysuccinimide, dichlorohexylcarbodiimide, N-beta-Maleimidopropionic acid, N-beta-maleimidopropyl succinimide ester or N-Succinimidyl 3-(2-pyridyldithio)propionate.

Further examples of covalent linkers and methods of making and using them are described in WO2016/205410, WO2018/145086, and WO 2020/180897, each of which is incorporated herein by reference.

Mono-Substituted Covalent Linker

The present disclosure relates to compounds (synthetic intermediates) comprising a homo-bivalent covalent linker substituted on one end by a substituent X, while remaining unsubstituted on the other end (a “mono-substituted covalent linker”), and wherein X comprises a biological moiety other than a nucleic acid. The mono-substituted covalent linker may be used, for example, as a synthetic intermediate in methods of making a variety of multi-conjugates as described below.

A variety of homo-bivalent linkers are known to those of skill in the art and are applicable to this disclosure, including but not limited to the homo-bivalent covalent linkers described herein.

A variety of biological moieties other than nucleic acids are known to those of skill in the art and are applicable to this disclosure as the substituent X, including but not limited to the non-nucleic acid biological moieties described herein.

In one aspect of the disclosure, the compound comprises a homo-bivalent covalent linker substituted on one end by a substituent X, wherein X comprises a biological moiety other than a nucleic acid, wherein the other end of the homo-bivalent linker is unsubstituted, and wherein the compound is at least 75% pure. In some aspects of the disclosure, the compound is at least 80, 85, 90, 95, 96, 97, 98, 99, or 100% pure. In some aspects, the compound is about 85% to 95% pure. In some aspects of the disclosure, preparations of the compound can be greater than or equal to 75% pure; greater than or equal to 85% pure; and greater than or equal to 95% pure.

In another aspect of the disclosure the compound comprises Structure 1:

X—R1-R2-A-R3-B (Structure 1)

wherein:
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
A is a group comprising the reaction product of a first nucleophile and a first electrophile;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide; and
B is a group comprising a second nucleophile or a second electrophile, wherein the second nucleophile is the same as the first nucleophile, and the second electrophile is the same as the first electrophile.

In some embodiments of the compound, the spacer group R2 comprises a C₂-C₁₀ alkyl, C₂-C₁₀ alkoxy, or C₁-C₁₀ aryl, or is absent.

In some embodiments of the compound, the first nucleophile and first electrophile of the group A comprise (i) a thiol and a maleimide, optionally wherein the reaction product of the thiol and maleimide is a derivative of succinamic acid (such as, when one of the maleimide rings is opened); (ii) a thiol and a vinylsulfone; (iii) a thiol and a pyridyldisulfide; (iv) a thiol and an iodoacetamide; (v) a thiol and an acrylate; (vi) an azide and an alkyne; or (vii) an amine and a carboxyl.

In some embodiments of the compound, A is a group comprising the reaction product of a thiol and a maleimide, optionally wherein the reaction product of the thiol and maleimide is a derivative of succinamic acid.

In some embodiments of the compound, the R3 group comprises a thiopropionate or disulfide.

In another aspect of the disclosure, the compound comprises Structure 2, or Structure 2a (representing two positional isomers):

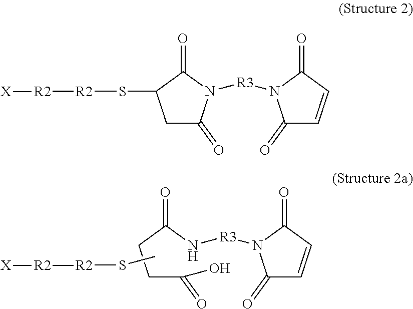

wherein:
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
S is sulfur;
N is nitrogen; and
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide.

In an embodiment of a compound comprising Structure 2, the R2 spacer group comprises a C₂-C₁₀ alkyl, C₂-C₁₀ alkoxy, or C₁-C₁₀ aryl.

In an embodiment of a compound comprising Structure 2:
X is a peptide or protein, or a derivative thereof;
R1 and R2 are absent; and
R3 is a group comprising a disulfide.

In an embodiment of a compound comprising Structure 2:
X is an organometallic compound, or a derivative thereof;
R1 is an ester group;
R2 is a spacer group comprising a C₂-C₁₀ alkyl; and
R3 is a group comprising a disulfide.

In an embodiment of a compound comprising Structure 2:
X is a small molecule, or a derivative thereof, such as a small molecule therapeutic drug;
R1 is an ester group;
R2 is a spacer group comprising a C₂-C₁₀ alkyl; and
R3 is a group comprising a disulfide.

In an embodiment of the compound, the homo-bivalent covalent linker comprises a linker that is cleavable under intracellular conditions.

In an embodiment of a compound comprising Structure 1 or Structure 2, the R3 group comprises a linker that is cleavable under intracellular conditions.

In an embodiment of the compound, substituent X is a peptide, protein, lipid, carbohydrate, carboxylic acid, vitamin, steroid, lignin, small molecule, organometallic compound, or a derivative of any of the foregoing.

In an embodiment of the compound, substituent X is (a) a peptide, or a derivative of a peptide, one example among many being the transduction domain of HIV-1TAT protein; (b) an antibody or antibody fragment, or a derivative thereof, an example of which is a single-chain variable fragment; (c) a carbohydrate, or a derivative of a carbohydrate; (d) a fatty acid, or a derivative of a fatty acid; (e) a vitamin, or a derivative of a vitamin, examples of which include but are not limited to tocopherol or folate; (f) a cholesterol, or a derivative thereof; (g) a carboxylic acid, or a derivative thereof, an example of which is (2S,2′S)-2,2′-(Carbonyldiimino)dipentanedioic acid (DUPA); (h) anisamide, or a derivative thereof; (i) an organometallic compound, or a derivative thereof, one example being ferrocene; (j) a small molecule, or a derivative thereof, one example being lenalidomide.

Methods for Synthesizing a Mono-Substituted Covalent Linker

The present disclosure provides methods for synthesizing a mono-substituted covalent linker comprising the steps of coupling a homo-bivalent covalent linker to a substituent X, wherein X is a biological moiety other than a nucleic acid, under reaction conditions that substantially favor the formation of the mono-substituted covalent linker and substantially prevent dimerization of the substituent X.

In some aspects of the disclosure, the method further comprises reacting the homo-bivalent covalent linker with a functionalized substituent X that comprises a functional group reactive with the homo-bivalent covalent linker. In an embodiment of this method, the functionalized substituent X comprises Structure 3:

X—R1-R2-A′ (Structure 3); and

the homo-bivalent covalent linker comprises Structure 4:

A″-R3-B (Structure 4); and
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide;
A′ and A″ together comprise a first nucleophile and a first electrophile that react together to form A; and
B is a group comprising a second nucleophile or a second electrophile, wherein the second nucleophile is the same as the first nucleophile, and the second electrophile is the same as the first electrophile; and
the resulting compound is Structure 1: X—R1-R2-A-R3-B.

In other aspects of the disclosure, the homo-bivalent covalent linker is reacted with a functionalized R2 end group to form an intermediate, and then the functionalized R2 end group of the intermediate is reacted with a functionalized substituent X that comprises a functional group reactive with the functionalized R2 end group to form R1; wherein: R1 is a group comprising phosphodiester, thiophosphodiester, sulfate, amide, triazole, heteroaryl, ester, ether, thioether, disulfide, thiopropionate, acetal, or glycol; and R2 is a spacer group. In an embodiment of this method, the homo-bivalent covalent linker comprises Structure 4:

A″-R3-B (Structure 4)

the functionalized R2 end group comprises Structure 5:

R1′-R2-A′ (Structure 5)

the functionalized substituent X comprises Structure 6:

X—R1″ (Structure 6); and
X is a substituent comprising a biological moiety other than a nucleic acid;
R1′ and R1″ are functional groups that react to form R1;
R1 is a group comprising phosphodiester, thiophosphodiester, sulfate, amide, triazole, heteroaryl, ester, ether, thioether, disulfide, thiopropionate, acetal, or glycol;
R2 is a spacer group;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide; and
A′ and A″ together comprise a first nucleophile and a first electrophile that react to form A;
B is a group comprising a second nucleophile or a second electrophile, wherein the second nucleophile is the same as the first nucleophile, and the second electrophile is the same as the first electrophile; and
the resulting compound is Structure 1: X—R1-R2-A-R3-B.

In an embodiment of the method, the coupling of the homo-bivalent covalent linker to the substituent X is carried out in a dilute solution of the functionalized substituent X with a stoichiometric excess of the homo-bivalent covalent linker.

In an embodiment of the method, the coupling of the homo-bivalent covalent linker to the substituent X is carried out with a molar excess of the homo-bivalent covalent linker of at least about 5, 10, 15, 20, 25, 30, 35, 40, 45, 50, or 100.

In an embodiment of the method, the coupling of the homo-bivalent covalent linker to the substituent X is carried out with a molar excess of the homo-bivalent covalent linker of about 5, 10, 15, 20, 25, 30, 35, 40, 45, 50, or 100.

In an embodiment of the method, the coupling of the homo-bivalent covalent linker to the substituent X is carried out in a solution comprising water and a water miscible organic co-solvent. In an embodiment, the water miscible organic co-solvent comprises DMF, NMP, DMSO, alcohol, or acetonitrile. In an embodiment, the water miscible organic co-solvent comprises about 10, 15, 20, 25, 30, 40, or 50% (v/v) of the solution.

Alcohols for use in the above-described methods of synthesis include but are not limited to C₁-C₁₀ alcohol, C₁-C₇ alcohol, and C₁-C₅ alcohol, in each case optionally substituted with a water miscible group such as amino, tertiary amino, or sulfate.

In an embodiment of the method, the coupling of the homo-bivalent covalent linker to the substituent X is carried out at a pH of below about 7, 6, 5, or 4.

In an embodiment of the method, the coupling of the homo-bivalent covalent linker to the substituent X is carried out at a pH of about 7, 6, 5, or 4.

In an embodiment of the method, the coupling of the homo-bivalent covalent linker to the substituent X is carried out in a solution comprising an anhydrous organic solvent. In a further embodiment, the anhydrous organic solvent comprises dichloromethane, DMF, DMSO, THF, dioxane, pyridine, alcohol, or acetonitrile.

In an embodiment of the method, the yield of the resulting mono-substituted covalent linker is at least 75, 80, 85, 90, 95, 96, 97, 98, 99, or 100%.

In an embodiment of the method, the purity of the resulting mono-substituted covalent linker is at least 75, 80, 85, 90, 95, 96, 97, 98, 99, or 100%. In some embodiments, the compound is about 85% to 95% pure. In some embodiments, preparations of the compound can be greater than or equal to 75% pure; greater than or equal to 85% pure; and greater than or equal to 95% pure.

Dimerical Multi-Conjugates

The present disclosure relates to multi-conjugates comprising a first substituent X, which comprises a biological moiety other than a nucleic acid and a second substituent Y that is the same or different from X, wherein X and Y are joined by a homo-bivalent covalent linker (a “dimerical multi-conjugate”). The disclosure is applicable to all types of homo-bivalent covalent linkers, cleavable or noncleavable, including, but not limited to, the examples of homo-bivalent covalent linkers provided herein; and further, to all types of biological moieties as the substituents of the dimerical multi-conjugate, non-nucleic in the case of X, and nucleic or non-nucleic in the case of Y, including, but not limited to, the various examples of substituents provided herein.

In some aspects of the disclosure, the multi-conjugate comprises Structure 7:

X●Y (Structure 7)

wherein:
X is a first substituent comprising a biological moiety other than a nucleic acid;
Y is a second substituent that is the same as or different from X; and
● is a covalent linker joining X and Y, and comprising Structure 8:

—R1-R2-A-R3-A-R2-R1— (Structure 8)
wherein:
   each R1 is independently a group comprising phosphodiester, thiophosphodiester, sulfate, amide, triazole, heteroaryl, ester, ether, thioether, disulfide, thiopropionate, acetal, glycol, or is absent;
   each R2 is independently a spacer group, or is absent;
   each A is the same and is a group comprising the reaction product of a nucleophile and an electrophile; and
   R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide.

In an embodiment of the multi-conjugate, Y is different from X.

In an embodiment of the multi-conjugate, X is a peptide, protein, lipid, carbohydrate, carboxylic acid, vitamin, steroid, lignin, small molecule, organometallic compound, or a derivative of any of the foregoing.

In an embodiment of the multi-conjugate, Y is a nucleic acid, peptide, protein, lipid, carbohydrate, carboxylic acid, vitamin, steroid, lignin, small molecule, organometallic compound, or a derivative of any of the foregoing.

In various embodiments of the multi-conjugate, X is: (a) a peptide or a derivative of a peptide, one example of which is the transduction domain of HIV-1TAT protein; (b) an antibody or an antibody fragment, or a derivative thereof, one example of which is an antibody single-chain variable fragment; (c) a small molecule, or a derivative thereof, such as a small molecule therapeutic drug, one example of which is lenalidomide; (d) an organometallic compound, or a derivative thereof, one example of which is ferrocene.

In various embodiments of the multi-conjugate, Y is nucleic acid, or a derivative thereof. In a further embodiment, Y is (a) RNA, examples of which include but are not limited to siRNA, saRNA, or miRNA; (b) an antisense oligonucleotide, examples of which include antisense DNA and gapmers; (c) a DNA or RNA aptamer, or (d) derivatives of any of the foregoing.

In an embodiment of the multi-conjugate, the homo-bivalent covalent linker joining X and Y is cleavable under intracellular conditions and may comprise any of the cleavable homo-bivalent covalent linkers disclosed herein or otherwise know to those of skill in the art.

In an embodiment, the multi-conjugate is at least 75, 80, 85, 90, 95, 96, 97, 98, 99, or 100% pure. In some aspects, the multi-conjugate is about 85% to 95% pure. In some embodiments, preparations of the multi-conjugate can be greater than or equal to 75% pure; greater than or equal to 85% pure; and greater than or equal to 95% pure.

Methods for Synthesizing Dimerical Multi-Conjugates

The present disclosure provides methods for synthesizing multi-conjugates comprising a first substituent X, which comprises a biological moiety other than a nucleic acid and a second substituent Y that is the same or different from X, wherein X and Y are joined by a homo-bivalent covalent linker (a dimerical multi-conjugate), under reaction conditions that substantially favor formation of the X●Y dimer and substantially prevent formation of the X●X dimer and the Y●Y dimer.

In an embodiment, the disclosure provides a method for synthesizing a multi-conjugate of Structure 7:

X●Y (Structure 7)

wherein:
X is a first substituent comprising a biological moiety other than a nucleic acid;
Y is a second substituent that is the same as or different from X; and
● is a covalent linker joining X and Y;

the method comprising the steps of:
(a) reacting X—R4 with a homo-bivalent linker ∘ to produce a mono-substituted product X-∘, wherein R4 is a functional group capable of reacting with ∘ under conditions that produce the mono-substituted product X-∘ and substantially prevent dimerization of X; and
(b) reacting X-∘ with R5-Y, wherein R5 is a functional group capable of reacting with ∘, thereby forming X●Y.

In another embodiment, the disclosure provides a method for synthesizing a multi-conjugate of Structure 7:

X●Y (Structure 7)

wherein:
X is a first substituent comprising a biological moiety other than a nucleic acid;
Y is a second substituent that is the same or different from X; and
is a covalent linker joining X and Y;

the method comprising the steps of:
(a) reacting R4-Y with a homo-bivalent linker ∘ to produce a mono-substituted product ∘-Y, wherein R4 is a functional group capable of reacting with ∘ under conditions that produce the mono-substituted product ∘-Y and substantially prevent dimerization of Y; and
(b) reacting ∘-Y with X—R5, wherein R5 is a functional group capable of reacting with ∘, thereby forming X●Y.

In an embodiment of the method for synthesizing a multi-conjugate of Structure 7, step (a) is carried out with a stoichiometric excess of the homo-bivalent linker ∘ relative to X—R4.

In an embodiment of the method for synthesizing a multi-conjugate of Structure 7, step (a) is carried out with a stoichiometric excess of the homo-bivalent linker ∘ relative to R4-Y.

In an embodiment of the method for synthesizing a multi-conjugate of Structure 7, step (a) is carried out with a molar excess of the homo-bivalent linker ∘ of about 5, 10, 15, 20, 25, 30, 35, 40, 45, 50, or 100.

In an embodiment of the method for synthesizing a multi-conjugate of Structure 7, step (a) is carried out in a solution comprising water and, optionally, a water miscible organic co-solvent. In a further embodiment, where a water miscible organic co-solvent is used, the water miscible organic co-solvent comprises DMF, DMSO, THF, dioxane, pyridine, alcohol, or acetonitrile. In a further embodiment, the water miscible organic co-solvent comprises about 10, 15, 20, 25, 30, 40, or 50% (v/v) of the solution

Alcohols for use in the above-described methods of synthesis include but are not limited to C₁-C₁₀ alcohol, C₁-C₇ alcohol, and C₁-C₅ alcohol, in each case optionally substituted with a water miscibility enhancing group such as amino, tertiary amino, or sulfate.

In an embodiment of the method for synthesizing a multi-conjugate of Structure 7, step (a) is carried out at a pH of about 7, 6, 5, or 4.

In an embodiment of the method for synthesizing a multi-conjugate of Structure 7, step (a) is carried out in a solution comprising an anhydrous organic solvent. In a further embodiment, the anhydrous organic solvent comprises dichloromethane, DMF, DMSO, THF, dioxane, pyridine, alcohol, or acetonitrile.

In an embodiment of the above-described methods: (a) the yield of the dimerical multi-conjugate is at least 75, 80, 85, 90, 95, 96, 97, 98, 99 or 100%; and/or (b) the purity of the dimerical multi-conjugate is at least 75, 80, 85, 90, 95, 96, 97, 98, 99 or 100%. In some embodiments, the multi-conjugate is about 85% to 95% pure. In some embodiments, preparations of the multi-conjugate can be greater than or equal to 75% pure; greater than or equal to 85% pure; and greater than or equal to 95% pure.

Larger Multi-Conjugates

The present disclosure also relates to larger multi-conjugates comprising at least three substituents, each of which is a biological moiety and may be the same or different, and each of which is joined to another by a covalent linker; wherein at least one substituent is not a nucleic acid (a non-nucleic acid substituent). The disclosure is applicable to all types of covalent linkers, cleavable or noncleavable, known to persons of ordinary skill in the art, including but not limited to the examples of covalent linkers provided herein; and further, to all types of biological moieties as substituents of the multi-conjugate, including but not limited to the various examples of substituents provided herein.

In various embodiments, the multi-conjugate comprises: (a) two or more substituents; (b) two, three, four, five, six, seven, eight, nine, or ten substituents.

In various embodiments, the multi-conjugate comprises: (a) at least two substituents that are different; (b) at least two substituents that are the same.

In some embodiments, (a) all of the substituents in the multi-conjugate are different; or (b) all of the substituents in the multi-conjugate are the same.

In an embodiment of this multi-conjugate, the disclosure provides a multi-conjugate comprising substituents X, Y and Z, wherein each of the substituents, independently, is a biological moiety and is joined to another substituent by a covalent linker ●; wherein the multi-conjugate comprises Structure 9:

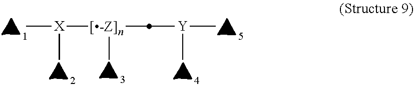

wherein:
each of ▴₁, ▴₂, ▴₃, ▴₄ and ▴₅ is, independently, absent or comprises a biological moiety covalently linked to its respective substituent;
n is an integer that is greater than or equal to zero; and
at least one of the substituents present in Structure 9 is not a nucleic acid.

In some embodiments of the multi-conjugate of Structure 9, n is 1, 2, 3, 4, 5, 6, 7, 8, 9, or 10.

In some embodiments of the multi-conjugate of Structure 9, X is not a nucleic acid. In some embodiments, Y is not a nucleic acid. In some embodiments, Z, in any of its iterations, is not a nucleic acid.

In an embodiment of the multi-conjugate of Structure 9, at least one covalent linker ● is a homo-bivalent covalent linker.

In an embodiment of the multi-conjugate of Structure 9, at least one ▴ is present. In some embodiments, at least one ▴ is a targeting moiety. In some embodiments, at least one ▴ is an endosomal escape moiety. In some embodiments, the multi-conjugate comprises at least one targeting moiety and at least one endosomal escape moiety. In some embodiments, at least one ▴ is a detectable label. In some embodiments, the multi-conjugate comprises at least one targeting moiety, endosomal escape moiety, and detectable label.

In an embodiment of the multi-conjugate of Structure 9, at least one terminus of the multi-conjugate is covalently bound to a ▴. In some embodiments, at least one internal substituent of the multi-conjugate is covalently bound to a ▴. In some embodiments, at least one terminus of the multi-conjugate is covalently bound to a ▴ and at least one internal substituent of the multi-conjugate is covalently bound to a ▴. In some embodiments, each of the termini of the multi-conjugate is covalently bound to a ▴ and each of the internal substituents of the multi-conjugate is covalently bound to a ▴.

In some embodiments, at least one of the ▴s that are present in the multi-conjugate is different from any other ▴ that is present in the multi-conjugate. In some embodiments, all of the ▴s that are present in the multi-conjugate are the same. In some embodiments, all of the ▴s that are present in the multi-conjugate are different.

In an embodiment of the multi-conjugate of Structure 9, at least one covalent linker ● is a sulfur-containing covalent linker; and at least one of ▴₁, ▴₂, ▴₃, ▴₄ and ▴₅ comprises a sulfur-containing end group Q. In a further embodiment, the sulfur-containing end group Q comprises a protected thiol group that is deprotectable under a deprotection condition; and the sulfur-containing covalent linker ● is stable under the deprotection condition. In another embodiment, the sulfur-containing covalent linker ● comprises a cleavable group that is cleavable under a cleavage condition that is not the deprotection condition. In a further embodiment, the sulfur-containing end group Q comprises a protected thiol.

In the various embodiments of the larger multi-conjugates, wherein at least one substituent is a non-nucleic acid substituent, there may be a stretch of two or more contiguous substituents that comprise nucleic acids; and this stretch is herein referred to as a “multimeric oligonucleotide component” of the multi-conjugate. Some multi-conjugates may contain more than one such multimeric oligonucleotide component.

A multimeric oligonucleotide component of a multi-conjugate thus comprises two or more oligonucleotides joined together by a covalent linker. The oligonucleotides may be single-stranded or double-stranded; and the oligonucleotides in the component may be the same (a homo-multimer) or different (a hetero-multimer). Examples of oligonucleotides that may make up a multimeric oligonucleotide component of a multi-conjugate include but are not limited to siRNA, saRNA, miRNA, DNA or RNA aptamers, and antisense oligonucleotides. In some embodiments, each oligonucleotide is 15-30, 17-27, 19-26, or 20-25 nucleotides in length.

A simple embodiment of a multimeric oligonucleotide component of a multi-conjugate is a dimer of two oligonucleotides, oriented 5′ to 3′, 3′ to 3′, or 5′ to 5′; each of which may be single-stranded or double-stranded; and wherein the sequence of each oligonucleotide may be the same or different.

In other embodiments of this aspect of the disclosure, the multimeric oligonucleotide component of a multi-conjugate comprises Structure 15:

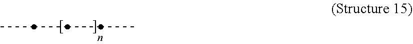

wherein each of the oligonucleotide subunits [symbol] is independently a single- or double-stranded oligonucleotide; each of the subunits [symbol] is joined to another subunit by a covalent linker ●; wherein at least one of the subunits [symbol] comprises a strand having one of the covalent linkers ● joined to its 3′ terminus and another of the covalent linkers joined to its 5′ terminus, and n is an integer≥0.

In other embodiments of this aspect of the disclosure, each oligonucleotide subunit [symbol] within a multimeric oligonucleotide component of a multi-conjugate is a double-stranded oligonucleotide [symbol] , and each covalent linker ● is on the same strand, as in Structure 16:

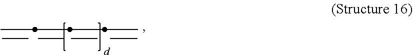

wherein d is an integer≥0.

In other embodiments of this aspect of the disclosure, each oligonucleotide subunit [symbol] within a multimeric oligonucleotide component of a multi-conjugate is a single-stranded oligonucleotide [symbol] , and each covalent linker ● is on the same strand, for example but not limited to a multimeric oligonucleotide of Structure 34:

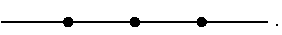

In one such embodiment, at least one single stranded oligonucleotide [symbol] is an antisense oligonucleotide. In another embodiment, each single stranded oligonucleotide [symbol] is independently an antisense oligonucleotide.

In other embodiments of this aspect of the disclosure, the multimeric oligonucleotide component comprises Structure 17 or Structure 18:

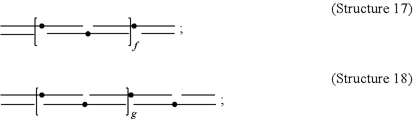

wherein each [symbol] is a double-stranded oligonucleotide, each ● is a covalent linker joining adjacent double-stranded oligonucleotides, f is an integer≥1, and g is an integer≥0.

In another embodiment of this aspect of the disclosure, the multimeric oligonucleotide component of the multi-conjugate comprises a compound according to Structures 19, 20, 21, or 22:

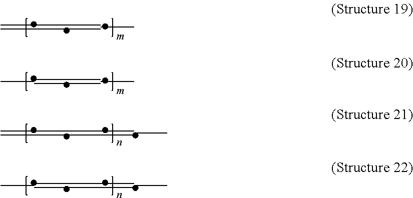

wherein:
each [symbol] is a double-stranded oligonucleotide,
each [symbol] is a single stranded oligonucleotide,
each ● is a covalent linker joining single strands of adjacent single stranded oligonucleotides, and m is an integer≥1 and n is an integer≥0.

In another embodiment of this aspect of the disclosure, the multimeric oligonucleotide component of the multi-conjugate comprises a branched structure wherein at least one of the covalent linkers ● joins three or more oligonucleotide subunits [symbol] . Structure 23 provides one example of a multimeric oligonucleotide component comprising a branched structure:

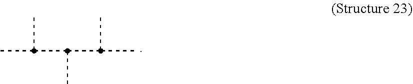

In yet another embodiment of this aspect of the disclosure, the multimeric oligonucleotide component of the multi-conjugate comprises an oligonucleotide subunit comprising a split strand, as in Structure 24:

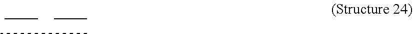

wherein:
each [symbol] is a partial single-stranded oligonucleotide; and
[symbol] is a complementary stand annealed to the partial single-stranded oligonucleotides.

In some embodiments of this aspect of the disclosure, each oligonucleotide subunit within a multimeric oligonucleotide component of a multi-conjugate independently contains fewer than 5 phosphorothioate groups; or fewer than 4 phosphorothioate groups; or fewer than 3 phosphorothioate groups.

In some embodiments of this aspect of the disclosure, fewer than 75% of the nucleotides within an oligonucleotide subunit are chemically modified; or fewer than 80% of the nucleotides within an oligonucleotide subunit are chemically modified.

In some embodiments of this aspect of the disclosure, at least one oligonucleotide subunit within an oligonucleotide component of a multi-conjugate is different from another subunit. In another embodiment, all of the oligonucleotide subunits within an oligonucleotide component are different from one another.

In some embodiments of this aspect of the disclosure, at least two oligonucleotide subunits within an oligonucleotide component of a multi-conjugate are joined by a covalent linker between the 3′ end of a first subunit and the 5′ end of a second subunit; or are joined by a covalent linker between the 5′ end of a first subunit and the 3′ end of a second subunit; or are joined by a covalent linker between the 5′ end of a first subunit and the 5′ end of a second subunit.

In some embodiments of this aspect of the disclosure, all double-stranded oligonucleotide subunits within in the oligonucleotide component of a multi-conjugate are blunt-ended (i.e., symmetrical); in other words, none contain strands with overhanging nucleotides (i.e., asymmetrical).

In some embodiments of this aspect of the disclosure, the multimeric oligonucleotide component of a multi-conjugate includes one or more chemically modified nucleotides but does not contain three identical chemical modifications on three consecutive nucleotides.

In some embodiments, the multimeric oligonucleotide component does not include a double-stranded subunit [symbol] having a sense and an antisense strand, wherein the sense and antisense strands comprise Structure F:

sense strand:
5′ nₚ-Nₐ-(XXX)ᵢ-N_{b}-YYY-N_{b}-(ZZZ)ⱼ-Nₐ-n_{q} 3′
antisense:
3′ nₚ′-Nₐ′-(X′X′X′)ₖ-N_{b}′-Y′Y′Y′-N_{b}′-(Z′Z′Z′)ₗ-Nₐ′-
n_{q}′ 5′

wherein i, j, k, and l are each independently 0 or 1; p, p′, q, and q′ are each independently 0-6; each Nₐ and Nₐ′ independently represents an oligonucleotide sequence comprising 0-25 modified nucleotides, each sequence comprising at least two differently modified nucleotides; each N_{b} and N_{b}′ independently represents an oligonucleotide sequence comprising 0-10 modified nucleotides; each nₚ′, nₚ, n_{q}′, and n_{q} independently represents an overhang nucleotide or may not be present; and XXX, YYY, ZZZ, X′X′X′, Y′Y′Y′, and Z′Z′Z′ each independently represent one motif of three identical modifications on three consecutive nucleotides. Each of X, Y and Z may be the same or different from each other.

In some embodiments, the multimeric oligonucleotide component does not include a double-stranded subunit [symbol] having a sense and an antisense strand, wherein the sense and antisense strands comprise Structure F1:

5′ nₚ-Nₐ-YYY-Nₐ-n_{q} 3′
3′ nₚ′-Nₐ′-Y′Y′Y′-Nₐ′-n_{q}′ 5′

wherein each Nₐ independently represents an oligonucleotide sequence comprising 2-20, 2-15, or 2-10 modified nucleotides.

In some embodiments, the multimeric oligonucleotide does not include a double-stranded subunit [symbol] having a sense and an antisense strand, wherein the sense and antisense strands comprise Structure F2:

5′ nₚ-Nₐ-YYY-N_{b}-ZZZ-Nₐ-n_{q} 3′
3′ nₚ′-Nₐ′-Y′Y′Y′-N_{b}′-Z′Z′Z′-Nₐ′-n_{q}′ 5′

each N_{b} independently represents an oligonucleotide sequence comprising 1-10, 1-7, 1-5 or 1-4 modified nucleo-tides. Each Nₐ independently represents an oligonucleotide sequence comprising 2-20, 2-15, or 2-10 modified nucleo-tides. Each of X, Y and Z may be the same or different from each other.

In some embodiments, the multimeric oligonucleotide component does not include a double-stranded subunit [symbol] having a sense and an antisense strand, wherein the sense and antisense strands comprise Structure F3:

5′ nₚ-Nₐ-XXX-N_{b}-YYY-Nₐ-n_{q} 3′
3′ nₚ′-Nₐ′-X′X′X′-N_{b}′-Y′Y′Y′-Nₐ′-n_{q}′ 5′

each N_{b}, N_{b}′ independently represents an oligonucleotide sequence comprising 0-10, 0-7, 0-10, 0-7, 0-5, 0-4, 0-2 or 0 modified nucleotides. Each Nₐ independently represents an oligonucleotide sequence comprising 2-20, 2-15, or 2-10 modified nucleotides. Each of X, Y and Z may be the same or different from each other.

In some embodiments, the multimeric oligonucleotide component does not include a double-stranded subunit [symbol] having a sense and an antisense strand, wherein the sense and antisense strands comprise Structure F4:

5′ nₚ-Nₐ-XXX-N_{b}-YYY-N_{b}-ZZZ-Nₐ-n_{q} 3′
3′ nₚ′-Nₐ′-X′X′X′-N_{b}′-Y′Y′Y′-N_{b}′-Z′Z′Z′-Nₐ′-n_{q}′ 5′

each N_{b}, N_{b}′ independently represents an oligonucleotide sequence comprising 0-10, 0-7, 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern. Each of X, Y and Z may be the same or different from each other.

In some embodiments, the multimeric oligonucleotide component does not include a double-stranded subunit [symbol] having a sense and an antisense strand, wherein the sense and antisense strands comprise Structure F5:

5′-Nₐ-Y Y Y-Nₐ-3′
3′ nₚ′-Nₐ′-Y′ Y′ Y′-Nₐ′ 5′

wherein:
nₚ′ is a 2-nucleotide overhang and each nucleotide within nₚ′ is linked to a neighboring nucleotide via a phosphorothioate linkage;
each Nₐ and Nₐ′ independently represents an oligonucleotide sequence comprising 0-25 nucleotides which are either modified or unmodified or combinations thereof, each sequence comprising at least two differently modified nucleotides;
YYY and Y′Y′Y′ each independently represent one motif of three identical modifications on three consecutive nucleotides.

In some embodiments, the multimeric oligonucleotide component does not include a double-stranded subunit represented by formula (I) in U.S. Pat. No. 10,612,024; US 2017/0275626 A1; US 2017/0369872 A1; WO 2019/036612 A1 in any of the various embodiments disclosed in these publications, each of which are incorporated herein in their entirety.

In some embodiments, the multimeric oligonucleotide component does not include a branched linkage coupling 3 or more oligonucleotides.

Further examples of multimeric oligonucleotides and methods for synthesizing multimeric oligonucleotides within the scope of this disclosure are disclosed in U.S. Pat. Nos. 9,644,209 and 10,597,659; WO 2016/205410 A2; WO 2018/145086 A1; and WO 2020/180897, each of which are incorporated herein in their entirety.

As described above, the covalent linkers in all of the multi-conjugates, some of which are represented as a “●”, may optionally include a spacer group Q on one or both sides of the covalent linkage, as in □-●, □-●-□, or ●-□.

Further, the covalent linkers in the multi-conjugates may be cleavable or noncleavable. Cleavable linkers may be selected or designed to maintain stability upon administration, while cleaving upon delivery, or under intracellular conditions, to facilitate functional delivery of the biological moieties. In addition to the examples of covalent linkers provided throughout this disclosure, those having ordinary skill will recognize that a wide variety of covalent linkers, including their composition, synthesis, and use are known in the art, and may be adapted for use consistent with this disclosure

In some embodiments of the larger multi-conjugates, each covalent linker is the same. In some embodiments, all of the covalent linkers are the different. In some embodiments, at least one of the covalent linkers is different from anther covalent linker.

In some embodiments, each covalent linker joins two substituents of the multi-conjugate. In some embodiments, at least one covalent linker joins three or more substituents of the multi-conjugate.

Methods for Synthesizing Larger Multi-Conjugates

The present disclosure also provides methods for synthesizing a multi-conjugate comprising biologically active substituents X, Y and Z, which may be the same or different, each joined to another by a covalent linker.

A method for synthesizing a multi-conjugate comprising Structure 10:

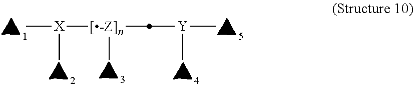

the method comprising:
reacting a compound of Structure 10a with a homo-bivalent linker to form a compound of Structure 10b under conditions that produce the mono-substituted product (Structure 10b) and substantially prevent dimerization of Structure 10a;
reacting the compound of Structure 10b with a compound of Structure 10c to form a compound of Structure 10d;
deprotecting the compound of Structure 10d to form a compound of Structure 10e; and
reacting the compound of Structure 10e with a compound of Structure 10f to form Structure 10; as follows:

▴₁-X-[●-Z′]ₙ′-R4 (Structure 10a)+homo-bivalent linker •

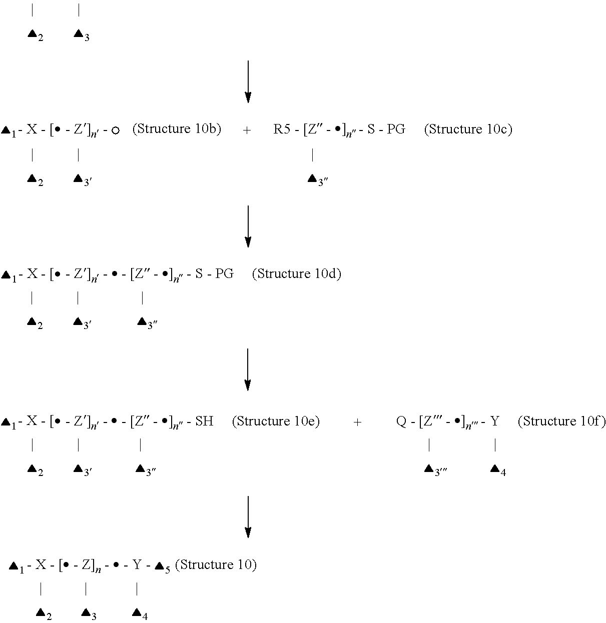

wherein:
● is a covalent linker;
∘ is a homo-bivalent linker;
R4 is a functional group selected to react with the homo-bivalent linker ∘ under conditions that produce the mono-substituted product of Structure 10b and substantially prevent dimerization of Structure 10a;
R5 is a functional group selected to react with the homo-bivalent linker ∘;
S-PG is a protected thiol group comprising a sulfur-containing group that is different from any sulfur-containing group residing within any of the covalent linkers ● within Structures 10b, 10c and 10d;
Q is a reactive group selected to react with the —SH group of Structure 10e to form a covalent linker ●;
X, Y, and Z are substituents of the multi-conjugate, and each is a biological moiety;
Z′, Z″, and Z′″ are substituents of the multi-conjugate, and each is a biological moiety;
each of ▴₁, ▴₂, ▴₃, ▴₄ and ▴₅ is, independently, absent or comprises a biological moiety covalently linked to its respective substituent;
each of ▴_{3′}, ▴_{3″}, and ▴_{3′″} is, independently, absent or comprises a biological moiety covalently linked to its respective substituent;
n is an integer that is greater than or equal to 1, and optionally n is 1, 2, 3, 4, 5, 6, 7, 8, 9, or 10; and
n′, n″, and n′″ are each an integer greater than or equal to zero, with the proviso that the sum of n′+n″+n′″ is n.

Methods for Using the Disclosed Compounds and Multi-Conjugates

The present disclosure also provides methods of using the disclosed multi-conjugates and synthetic intermediates in modulating gene expression, biological research, treating or preventing medical conditions, and/or to produce new or altered genotypes or phenotypes.

Pharmacokinetic and Pharmacodynamic Properties of the Multi-Conjugates

The bioavailability of a drug in the blood stream can be characterized as the balance between target cell uptake versus kidney clearance. From a practical perspective, in vivo circulation half-life and/or in vivo activity are good proxies for kidney clearance/glomerular filtration because they can be readily quantified and measured and because their improvement (e.g., increase) can correlate with improved pharmacodynamics and/or pharmacokinetics.

The uptake rate of a therapeutic agent (TA) in the blood is a function of a number of factors, which can be represented as: Rate of Uptake=f {(TA Concentration)×(Rate Blood Flow)×(Receptor Copy Number/cell)×(Number of Cells)×(equilibrium dissociation constant K_{d})×(Internalization Rate)}. For a given ligand/receptor pair, the Copy Number, K_{d}, Number of Cells and Internalization Rate will be constant. This can explain why the GalNAc ligand system is so effective for hepatocytes—it targets the ASGP receptor, which is present at high copy number and has a high internalization rate. The K_{d} of some ASGP/GalNAc variants is in the nanomolar range and the internalization rate is very high.

However, effective targeting is also dependent on the concentration of the therapeutic agent, which rapidly decreases over time due to clearance from the blood stream. The rate of clearance of a therapeutic agent (TA) can be represented as: Rate of Clearance=f {(Blood Flow Rate)×(Kidney Filtration Rate)×(Other clearance mechanisms)}. The resulting concentration of TA at time t can be represented as: (ONT Concentration)t=f {(Initial Concentration)−(Rate of Clearance×t)}.

In humans, clearance is mainly due to glomerular filtration in the kidney. In general, molecules less than about 45 kD have a half-life of about 30 minutes. In mice, the rate of clearance is even faster, the circulation half-life being about 5 minutes. Without wishing to be bound by any particular theory, it is believed that the disclosure can reduce glomerular filtration using specifically configured multi-conjugates (e.g., specific composition, size, weight, etc.), leading to a lower rate of clearance, resulting in a higher concentration of therapeutic agent(s) in circulation at a given time t (e.g., increased serum half-life, higher overall uptake, and higher activity).

Again, without wishing to bound by any particular theory, actual glomerular filtration rates can be difficult to measure directly. For example, compounds that pass through the glomerular capillaries are readily absorbed by cells such as tubular epithelial cells, which can retain therapeutic agents or their metabolites for significant periods of time (see, e.g., Henry, S. P. et al; Toxicology, 301, 13-20 (2012) and van de Water, F. M et al; Drug metabolism and Disposition, 34, No 8, 1393-1397 (2006)). In addition, absorbed compounds can be metabolized to breakdown products, which are then excreted in urine. Thus, the concentration (e.g., in urine) of a therapeutic agent at a specific time point may not necessarily be representative of the glomerular filtration rate. However, serum half-life, which is related to glomerular filtration and which is directly measurable, may be considered to be a suitable proxy for glomerular filtration.

Table 1 below shows the dramatic effect increasing the circulation half-life (t_{1/2}) of a component can have on the resulting concentration of the component at time t.

TABLE 1
Effect of increasing circulation half-
life (t_{1/2}) on concentration at time t.

| t (min): | 0 | 30 | 60 | 90 | 120 | 150 | 180 | 210 | 240 |
|---|---|---|---|---|---|---|---|---|---|
| 30 min t_{1/2} | 100 | 50 | 25 | 12.5 | 6.25 | 3.13 | 1.56 | 0.78 | 0.4 |
| 60 min t_{1/2} | 100 | | 50 | | 25 | | 12.5 | | 6.25 |
| 90 min t_{1/2} | 100 | | | 50 | | | 25 | | |
| 120 min t_{1/2} | 100 | | | | 50 | | | | 25 |

Values are presented as % initial dose at time t.

Thus, increasing the half-life of a component by a factor of two increases its residual concentration at two hours by a factor of four. Increasing the half-life by a factor of four leads to even more dramatic improvements in residual concentration—by factors of eight and greater than sixty at two and four hours, respectively.

The multi-conjugates disclosed herein may be configured to have a molecular weight and/or size and/or structure that will produce decreased clearance of the multi-conjugate by the kidney and/or other routes of clearance. This configuration strategy may also result in increased circulation half-life of the multi-conjugate and/or increased bioactivity of one or more substituents within the multi-conjugate when administered to a subject, in each case relative to the circulation half-life and bioactivity, respectively, of the same substituent when administered in monomeric form.

In the case of an siRNA substituent in a multi-conjugate, for example, increased bioactivity may be represented by the measurement of lower levels of the siRNA's target protein or mRNA after administration of the multi-conjugate relative to levels of the same protein or mRNA when measured after administration of a corresponding monomeric siRNA oligonucleotide.

In various aspects of the disclosure, the in vivo circulation half-life or serum half-life increases by a factor of at least 2, 3, 4, 5, 6, 7, 8, 9, 10, 20, 30, 40, 50, 60, 70, 80, 90, 100, 150, 200, 250, 500, or 1,000. In various aspects, the increase in bioactivity is measured as the ratio of bioactivity at tₘₐₓ. In various embodiments, the bioactivity increases by a factor of at least 2, 3, 4, 5, 6, 7, 8, 9, 10, 20, 30, 40, 50, 60, 70, 80, 90, 100, 150, 200, 250, 500, or 1,000. The relative increase in bioactivity of at least one of the substituents in the multimer as compared to its corresponding monomeric form may be in the range of greater than or equal to 2-10 times higher; for example, the relative increase may be 2, 5, 10, or more times that of the corresponding monomer. In one embodiment, these increases are observed in a mouse. In another embodiment, these increases are observed in a human.

In some aspects of the disclosure, the multi-conjugate is configured to have a molecular weight of at least about 45 kD, or a molecular weight in the range of about 45 kD to 60 kD.

In an embodiment, the increase in serum half-life and/or bioactivity of one or more substituents within the multi-conjugate is independent of phosphorothioate content in the multi-conjugate.

In a further aspect, the multi-conjugate, when administered to a subject via subcutaneous (SC) administration, may have a reduced rate of release from the SC tissue relative to monomeric forms of one or more of the substituents. When the aspects of SC administration and increased serum half-life of a multi-conjugate are combined, there may be a synergistic effect on bioactivity resulting from the multi-conjugate's reduced rate of release from the SC tissue coupled with reduced systemic clearance via the kidney, thereby further increasing the potential over time for cellular delivery and internalization of the multi-conjugate relative to monomer equivalent, and thereby further increasing bioactivity of at least one substituent in the multi-conjugate relative to its monomeric equivalent.

When combined with a cell- or tissue-targeting moiety, a multi-conjugate comprising two or more substituents of the same active agent can deliver a higher payload per cell or tissue binding event than the monomeric equivalent of the active agent. The multi-conjugate can also comprise two or more copies of a targeting moiety. Likewise, the multi-conjugate can additionally comprise other biological moieties designed for other purposes, such as to expedite functional delivery or intracellular release. In combination, these effects can lead to a dramatic increase in the uptake and bioactivity of the therapeutic agent. This can be advantageous where some combination of the receptor copy number, Kd, number of target cells and internalization rate of a given ligand/receptor pair is sub-optimal.

Certain polymer linking agents and multi-conjugate substituents such as polyethylene glycol (PEG) may be useful for increasing the circulation half-life of certain drugs. Such approaches can have drawbacks, including “diluting” the therapeutic agent (e.g., less active agent per unit mass). The present disclosure, in some aspects, is distinguished from such approaches. For example, in various embodiments, the multi-conjugate does not comprise PEG or analogous constituents. In various embodiments, the multi-conjugate does not comprise a polyether compound. In various embodiments, the multi-conjugate does not comprise a polymer other than, in some instances, oligonucleotides.

Nanoparticles, such as lipid nanoparticles (LNPs) have been used in attempts to increase the circulation half-life of certain drugs. Such approaches can have drawbacks, including increased toxicity (e.g., from cationic lipids). The present disclosure can be distinguished from such approaches. For example, in various aspects of the disclosure, the multi-conjugate is not formulated in a nanoparticle.

In addition, phosphorothioate groups have been used to increase the circulation half-life of certain drugs. Such approaches can have the drawbacks, including lower activity. The present disclosure can be distinguished from such approaches. For example, in various aspects of the disclosure, the multi-conjugate does not comprise a phosphorothioate or contains a smaller number of phosphorothioates as compared to analogous multi-conjugates known in the field.

Accordingly, the multi-conjugates disclosed herein may be configured in terms of molecule size, weight and/or structure to (a) increase in vivo circulation half-life of the multi-conjugate relative to that of one or more of the individual monomeric substituents and/or (b) increase bioactivity of the multi-conjugate relative to that of one or more individual monomeric substituents.

Methods of Treatment and Methods of Administration

In various aspects, the disclosure provides methods of treating a subject desiring prophylaxis or amelioration of a disease or disorder administering an effective amount of a multi-conjugate of the present disclosure to the subject. In such methods, the multi-conjugate comprises two or more substituents covalently linked together, each substituent comprising a biological moiety, wherein at least one substituent is not a nucleic acid (i.e., is a “non-nucleic acid substituent”).

In some methods of treatment, the multi-conjugate has three or more substituents; or the multi-conjugate has 3, 4, 5, 6, 7, 8, 9, 10, 11 or 12 substituents.

In some methods of treatment, the multi-conjugate has at least one covalent linker that is a homo-bivalent covalent linker.

In some methods of treatment, the multi-conjugate comprises at least one covalent linker that is cleavable under intracellular conditions. In some methods, the multi-conjugate may comprise any of the cleavable homo-bivalent covalent linkers disclosed herein or otherwise know to those of skill in the art.

Examples of non-nucleic acid substituents include but are not limited to peptides, proteins, lipids, carbohydrates, carboxylic acids, vitamins, steroids, lignins, small molecules, organometallic compounds, or derivatives of any of the foregoing.

Examples of nucleic acid substituents include but are not limited to DNA and RNA, double-stranded or single-stranded, including natural and synthetic derivatives thereof. In some instances, one or more substituents in the multi-conjugate comprise siRNA, saRNA, or miRNA. In some instances, one or more substituents in the multi-conjugate comprise an antisense oligonucleotide. In some instances, one or more substituents in the multi-conjugate comprise DNA or RNA aptamers.

In some methods of treatment, the multi-conjugate has a molecular weight and/or size configured to increase serum half-life of the multi-conjugate and/or in vivo activity of one or more substituents of the multi-conjugate; in each case relative to the serum half-life or in vivo activity of the same substituent when administered in monomeric form.

In some methods of treatment described herein, the multi-conjugate has a molecular weight and/or size configured to decrease clearance of the multi-conjugate via the kidney. In some embodiments, the decreased clearance of the multi-conjugate via the kidney may be a result of decreased glomerular filtration.

In one embodiment of such a method, the decreased clearance of the multi-conjugate via the kidney is determined by measuring the in vivo circulation half-life of the multi-conjugate after administering the multi-conjugate to the subject.

In another embodiment, the decreased clearance of the multi-conjugate via the kidney is determined by measuring the time required for the serum concentration of the multi-conjugate to decrease to a predetermined value. The predetermined value can be 90%, 80%, 70%, 60%, 55%, 50%, 45%, 40%, 35%, 30%, 25%, 20%, 15%, 10%, 5%, 4%, 3%, 2%, or 1% of the administered dose.

In another embodiment, the decreased clearance via the kidney is determined by measuring the serum concentration of the multi-conjugate at a predetermined time after administering the multi-conjugate to the subject.

In another embodiment, the decreased clearance via the kidney is determined by measuring the area under a curve of a graph representing serum concentration of the multi-conjugate over time after administering the multi-conjugate to the subject.

In some methods of treatment described herein, the decreased clearance of the multi-conjugate results in increased in vivo bioavailability of the multi-conjugate in the subject to which it is administered. For example, in one embodiment, the increased bioavailability of the multi-conjugate results in an increase in in vivo cellular uptake of the multi-conjugate. In another embodiment, the increased bioavailability of the multi-conjugate results in an increase in the in vivo therapeutic index/ratio of the multi-conjugate. In yet another embodiment, the increased bioavailability of the multi-conjugate results in an increase in the in vivo bioactivity of at least one substituent of the multi-conjugate relative to a corresponding monomeric form of the substituent. And indeed, each of these embodiments may occur alone or together with one or more additional embodiments as a result of administration of the multi-conjugate in a method of treating a subject.

In one aspect of these methods, a measured parameter relating to decreased clearance of the multi-conjugate via the kidney, for example serum half-life of the multi-conjugate has a sigmoidal relationship with respect to the number of substituents in a monomeric, dimeric, trimeric and higher number multi-conjugate.

In one aspect of these methods, the measured parameter for the multi-conjugate and each of its substituents starting with a monomeric substituent, when plotted, define a sigmoidal curve.

The disclosure further provides a method of administering a multimeric oligonucleotide to a subject in need thereof, wherein the number of substituents contained in the multi-conjugate is m, m being an integer selected to enable the multi-conjugate to have a molecular weight and/or size configured to increase the serum half-life and/or in vivo activity of one or more substituents within the multi-conjugate relative to the serum half-life and/or in vivo activity of the same substituent when administered in monomeric form. In various aspects, m is ≥2, ≥3, ≥4, ≥4 and ≤17, ≥4 and ≤8, or 3, 4, 5, 6, 7, or 8.

In some methods of administration disclosed herein, the molecular weight of the multi-conjugate administered to a patient is at least about 45 kD, or in the range of about 45 kD to 60 kD.

In one aspect, the disclosure provides a method for delivering two or more therapeutic agents to a cell per targeting ligand binding event comprising administering an effective amount of a multi-conjugate according to the disclosure to a subject in need thereof, wherein the multi-conjugate comprises a targeting ligand.

In one aspect, the disclosure provides a method for delivering a predetermined stoichiometric ratio of two or more therapeutic agents to a cell comprising administering an effective amount of a multi-conjugate according to the disclosure to a subject in need thereof, wherein the multi-conjugate comprises the predetermined stoichiometric ratio of two or more therapeutic agents.

In various embodiments of the methods or treatment and methods of administration described herein, the administered multi-conjugate is least 75, 80, 85, 90, 95, 96, 97, 98, 99, or 100% pure. In some aspects, the multi-conjugate is about 85% to 95% pure. In some embodiments, preparations of the multi-conjugate can be greater than or equal to 75% pure; greater than or equal to 85% pure; and greater than or equal to 95% pure.

Methods for Treating Genetic or Oncogenetic Disease and for Modulating Gene Expression

The disclosure provides methods for using the disclosed multi-conjugates for the treatment of diseases or disorders that may be addressed by modulating gene expression, for example by up-regulating, or down-regulating or silencing gene expression, or by affecting mRNA splicing. A large variety of diseases and disorders of this nature are known, as are the biological moieties that may be selected or developed to address the condition. The teachings of this disclosure will serve to enable persons of skill to design and develop multi-conjugates for the medical or veterinary treatment of such conditions, or for modulating gene expression in other fields such as basic research, agriculture, diagnostics and animal husbandry. Where multiple therapeutic or other targets are desired to be affected, the multi-conjugates of the present disclosure enable multi-targeting to be achieved with a single chemical entity.

In one aspect, the disclosure provides a method for treating a subject with a disease or disorder that would benefit from modulating gene expression, the method comprising administering an effective amount of a multi-conjugate according to the disclosure to the subject.

In one aspect, the disclosure provides a method for modulating gene expression of a target gene, comprising administering an effective amount of a multi-conjugate according to the disclosure to a subject in need thereof. In such therapeutic embodiments, the multi-conjugate will comprise at least one substituent that modulates gene expression, for example but not limited to an siRNA, a miRNA, a saRNA, or an antisense oligonucleotide, a CRISPR nuclease, a crRNA, and derivatives of any of the foregoing.

Similarly, the disclosure provides a method for modulating expression of two or more target genes comprising administering an effective amount of a multi-conjugate according to the disclosure to a subject in need thereof, wherein the multi-conjugate comprises substituents that modulate gene expression in the two or more target genes, for example but not limited to an siRNA, a miRNA, a saRNA, or an antisense oligonucleotide a CRISPR nuclease, a crRNA, and derivatives of any of the foregoing. The multi-conjugate can comprise substituents targeting two, three, four, or more genes.

In all of the foregoing aspects of the disclosure, the multi-conjugate may include, in addition to the one or more substituents that modify gene expression, other substituents that produce other therapeutic effects, including but not limited to immune stimulation or suppression, check point inhibition, and inflammation reduction. Multi-conjugates comprising substituents that provide multi-therapeutic effects will serve to advance treatments for complex diseases and conditions such as cancer, autoimmune and neurological disorders.

Subjects

Subjects that may benefit from the disclosed methods of treatment and methods of administration disclosed herein include, but are not limited to, mammals, such as primates, rodents, and agricultural animals. Examples of primate subjects include, but are not limited to, humans, chimpanzees, and macaques. Examples of a rodent subject includes, but is not limited to, a mouse and a rat. Examples of an agricultural animal subject includes, but is not limited to, a cow, a sheep, a lamb, a chicken, and a pig.

Mouse glomerular filtration rate (GFR) can be about 0.15 ml/min.-0.25 ml/min. Human GFR can be about 1.8 ml/min/kg (Mahmood I: (1998) Interspecies scaling of renally secreted drugs. Life Sci 63:2365-2371).

Mice can have about 1.46 ml of blood. Therefore, the time for glomerular filtration of total blood volume in mice can be about 7.3 minutes (1.46/0.2). Humans can have about 5 liters of blood and weigh about 70 kg. Therefore, the time for glomerular filtration of total blood volume in humans can be about 39.7 mins [5000/126(1.8*70)].

A person of ordinary skill in the art would recognize that different species can have different rates of clearance by glomerular filtration, at least for the above reasons. A person of ordinary skill in the art can infer that a ratio of rate of clearance by glomerular filtration between human and mouse times can be about 1:5 or 1:6. In other words, the rate of clearance of a certain substance by humans can be 5-6 times slower than that of a mouse.

In one aspect, the disclosure provides a method of delivering a multi-conjugate to a subject in need thereof, wherein the in vivo circulation half-life is measured between 30 minutes and 120 minutes after delivering the multimeric oligonucleotide to the subject.

In one aspect, the disclosure provides a method of delivering a multi-conjugate to a subject in need thereof, wherein the predetermined time is between 30 minutes and 120 minutes after delivering the multi-conjugate to the subject.

In one aspect, the disclosure provides a method of delivering a multi-conjugate to a subject in need thereof, wherein the area under the curve is calculated based on serum concentration of the multi-conjugate between x and y minutes after administering the multi-conjugate to the subject. In some embodiments, x can be 1, 2, 3, 4, 5, 10, 20, 30, 40, 50, 60, 75, 90, 120, 180, 240, or 300 minutes and y can be 90, 120, 180, 240, 300, 360, 420, 480, 540, 600, 720, 840, 960, 1080, 1200, 1320, 1440, or 1600 minutes. For example, the time range can be about 30 minutes-120 minutes, about 1 minute-1600 minutes, or about 300 minutes-600 minutes.

In one aspect, the disclosure provides a multi-conjugate or a method for increasing in vivo circulation half-life of the multi-conjugate, wherein the multi-conjugate is not formulated in a nanoparticle (NP) or a lipid nanoparticle (LNP).

In one aspect, the disclosure provides a multi-conjugate or a method for increasing in vivo circulation half-life of the multi-conjugate that is not dependent upon aggregation of the multi-conjugate with endogenous serum proteins.

In this, and other embodiments, the multi-conjugates of the disclosure can be administered in the form of a pharmaceutical composition, in a delivery vehicle, or coupled to a targeting ligand.

Pharmaceutical Compositions

In various aspects, the disclosure provides pharmaceutical compositions including any one or more of the multi-conjugates described herein. As used herein, pharmaceutical compositions include active agents, other than foods, that can be used to prevent, diagnose, alleviate, treat, or cure a disease. Similarly, the various multi-conjugates according to the disclosure should be understood as including embodiments for use as a medicament and/or for use in the manufacture of a medicament.

A pharmaceutical composition can include a multi-conjugate according to the disclosure and a pharmaceutically acceptable excipient. As used herein, an excipient can be a natural or synthetic substance formulated alongside the active ingredient. Excipients can be included for the purpose of long-term stabilization, increasing volume (e.g., bulking agents, fillers, or diluents), or to confer a therapeutic enhancement on the active ingredient in the final dosage form, such as facilitating drug absorption, reducing viscosity, or enhancing solubility. Excipients can also be useful in manufacturing and distribution, for example, to aid in the handling of the active ingredient and/or to extend shelf-life stability (e.g., by preventing denaturation or aggregation). As will be understood by those skilled in the art, appropriate excipient selection can depend upon various factors, including the route of administration, dosage form, and active ingredient(s).

Multi-conjugates can be administered in a variety of ways, including but not limited to locally or systemically, and thus the pharmaceutical compositions of the disclosure can vary accordingly. Administration is not limited to any particular delivery route, system, or technique and may include, without limitation, parenteral (including subcutaneous, intravenous, intramedullary, intraarticular, intramuscular, intraperitoneal, intraocular, or CNS injection), rectal, topical, transdermal, oral, or by inhalation (intranasally or to the lungs by way of, e.g., a nebulizer). Administration to an individual may occur in a single dose or in repeat administrations, and in any of a variety of physiologically acceptable salt forms, and/or with an acceptable pharmaceutical carrier and/or additive as part of a pharmaceutical composition. Physiologically acceptable formulations and standard pharmaceutical formulation techniques, dosages, and excipients are well known to persons skilled in the art (see, e.g., Physicians' Desk Reference (PDR®) 2005, 59th ed., Medical Economics Company, 2004; and Remington: The Science and Practice of Pharmacy, eds. Gennado et al. 21th ed., Lippincott, Williams & Wilkins, 2005).

Pharmaceutical compositions include an effective amount of the multi-conjugate according to the disclosure. As used herein, “effective amount” can be a concentration or amount that results in achieving a particular purpose, for example, an amount adequate to cause a biological effect, for example in comparison to a placebo. Where the effective amount is a “therapeutically effective amount,” it can be an amount adequate for therapeutic use, for example an amount sufficient to prevent, diagnose, alleviate, treat, or cure a disease. An effective amount can be determined by methods known in the art. For example, a therapeutically effective amount can be determined empirically, for example by human clinical trials. Effective amounts can also be extrapolated from one animal (e.g., mouse, rat, monkey, pig, dog) for use in another animal (e.g., human), using conversion factors known in the art. See, e.g., Freireich et al., Cancer Chemother Reports 50(4):219-244 (1966).

Delivery Constructs and Formulations

As will be understood by those skilled in the art, regardless of biological target or mechanism of action, therapeutic multi-conjugates must overcome a series of physiological hurdles to access a target cell or tissue in an organism (e.g., animal, such as a human, in need of therapy). Therapeutic multi-conjugates generally must avoid clearance in the bloodstream, enter the target cell type, and enter the cytoplasm, and sometimes enter the nucleus, all without eliciting an undesirable immune response. In various aspects, the disclosure provides multi-conjugates for direct delivery to cell and tissue targets. Alternatively, the disclosure provides for multi-conjugates formulated in a delivery vehicle.

In direct delivery strategies, the multi-conjugates will, in most instances, require stabilization, usually in the form of chemical modification of the substituents, to enable them to withstand degradation by serum nucleases and other factors, and to avoid the triggering of an innate immune response. Chemical stabilization strategies are known to those of skill in the art and may be readily used or adapted in connection with the multi-conjugates disclosed herein. Alternatively, when formulated in a delivery vehicle or formulation, such as a lipid nanoparticle (LNP), exosome, microvesicle, or viral vector, multi-conjugates may be delivered without chemical modification, or with minimal modification, as they may be protected or masked from degradation and immune activity by the delivery vehicle. Delivery vehicles and formulations are known to those of skill in the art and may be readily used or adapted in connection with the multi-conjugates disclosed herein.

In some aspects of the disclosure, multi-conjugates are equipped with a targeting moiety, such as a cell- or tissue-targeting ligand for direct delivery to a target cell or tissue without the need for formulation in a delivery vehicle. In other embodiments, where the multi-conjugate is formulated in a delivery vehicle, the delivery vehicle may be equipped with a cell- or tissue-targeting moiety. Examples of targeting moieties suitable for use in connection with this disclosure include but are not limited to a lipophilic moiety (e.g., a phospholipid); aptamers; peptides or proteins (e.g., arginine-glycine-aspartic acid [RGD], transferrin, monoclonal antibodies or fragments thereof, such as a single chain variable fragment (ScFv), or a VHH antigen-binding protein); cell growth factors, small molecules, vitamins (e.g., folate, tocopherol), carbohydrates (e.g., monosaccharides and polysaccharides, N-Acetylgalactosamine [GalNAc], galactose, mannose); cholesterol; glutamate ureas (e.g., 2-[3-(1,3-dicarboxypropyl)-ureido]pentanedioic acid [DUPA]), benzamide derivatives (e.g., anisamide); and derivatives of any of the foregoing. In some embodiments, the GalNac targeting moiety may be a mono-antennary GalNAc, a di-antennary GalNAc, or a tri-antennary GalNAc.

The lipophilic moiety may be a ligand that includes a cationic group. In certain embodiments, the lipophilic moiety is a cholesterol, vitamin E, vitamin K, vitamin A, folic acid, or a cationic dye (e.g., Cy3). Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

The targeting moiety may be a fatty acid, such as cholesterol, Lithocholic acid (LCA), Eicosapentaenoic acid (EPA), Docosahexaenoic acid (DHA), and Docosanoic acid (DCA), steroid, secosteroid, lipid, ganglioside or nucleoside analog, endocannabinoid, and/or vitamin such as choline, vitamin A, vitamin E, and derivatives or metabolites thereof, or a vitamin such as retinoic acid and alpha-tocopheryl succinate.

A targeting moiety may be incorporated into a multi-conjugate using the teachings of this disclosure as well as other techniques known in the art, including but not limited to a covalent bond, an amide bond, or an ester bond, or via a non-covalent bond such as biotin-streptavidin, or a metal-ligand complex.

A targeting moiety can be bound to the multi-conjugate at a terminal location, or in some instances, at an internal location. In some embodiments, two targeting moieties are incorporated into the multi-conjugate, e.g., where a targeting moiety is conjugated at each terminus of the multi-conjugate. More than two targeting moieties may be incorporated into the multi-conjugate, if desired, and at a variety of locations both internal and terminal.

In various aspects, the disclosure provides for the use and incorporation of endosomal escape moieties (EEMs) to facilitate endosomal escape of a multi-conjugate that has been endocytosed by a cell. Endosomal escape moieties are generally lipid-based or amino acid-based, but may comprise other chemical entities that disrupt an endosome to release the multi-conjugate or its metabolites. Examples of EEMs include, but are not limited to, chloroquine, peptides and proteins with motifs containing hydrophobic amino acid R groups, and influenza virus hemagglutinin (HA2). Further EEMs are described in Lonn et al., Scientific Reports, 6: 32301, 2016.

In various aspects, the disclosure provides for the use and incorporation of nuclear localization signals or sequences (NLS) to facilitate importation of the multi-conjugate, or a portion thereof (e.g., a substituent of the multi-conjugate liberated by cleavage of a linker), to the nucleus of a cell to which the multi-conjugate has been delivered. The NLS is typically an amino acid sequence, examples of which are known by those working in the field of drug delivery.

Numerous drug delivery vehicles have been designed to overcome the obstacles of in vivo delivery. These vehicles have been used to deliver therapeutic RNAs, small molecule drugs, protein drugs, and other therapeutic molecules. Drug delivery vehicles have been made from materials as diverse as sugars, lipids, lipid-like materials, proteins, polymers, peptides, metals, hydrogels, conjugates, and peptides. Many drug delivery vehicles incorporate aspects from combinations of these groups, for example, some drug delivery vehicles can combine sugars and lipids. In some other examples, drugs can be directly hidden in “cell like” materials that are meant to mimic cells, while in other cases, drugs can be put into, or onto, cells themselves. Drug delivery vehicles can be designed to release drugs in response to stimuli such as pH change, biomolecule concentration, magnetic fields, and heat.

In some aspects of the disclosure, the multi-conjugates can be encapsulated in a carrier material to form nanoparticles for intracellular delivery. Known carrier materials include cationic polymers, lipids or peptides, or chemical analogs thereof. Jeong et al., BIOCONJUGATE CHEM., Vol. 20, No. 1, pp. 5-14 (2009). Examples of a cationic lipid include dioleyl phosphatidylethanolamine, cholesterol dioleyl phosphatidylcholine, N-[1-(2,3-dioleoyloxy)propyl]-N,N,N-trimethylammonium chloride (DOTMA), 1,2-dioleoyloxy-3-(trimethylammonio)propane (DOTAP), 1,2-dioleoyl-3-(4′-trimethyl-ammonio)butanoyl-sn-glycerol (DOTB), 1,2-diacyl-3-dimethylammonium-propane (DAP), 1,2-diacyl-3-trimethylammonium-propane (TAP), 1,2-diacyl-sn-glycerol-3-ethylphosphocholin, 3 beta-[N—(N′,N′-dimethylaminoethane)-carbamoyl]cholesterol (DC-Cholesterol), dimethyldioctadecylammonium bromide (DDAB), and copolymers thereof. Examples of a cationic polymer include polyethyleneimine, polyamine, polyvinylamine, poly(alkylamine hydrochloride), polyamidoamine dendrimer, diethylaminoethyl-dextran, polyvinylpyrrolidone, chitin, chitosan, and poly(2-dimethylamino)ethyl methacrylate. In one embodiment, the carrier contains one or more acylated amines, the properties of which may be better suited for use in vivo as compared to other known carrier materials.

In one aspect of the disclosure, the carrier is a cationic peptide, for example KALA (a cationic fusogenic peptide), polylysine, polyglutamic acid or protamine. In another aspect, the carrier is a cationic lipid, for example dioleyl phosphatidylethanolamine or cholesterol dioleyl phosphatidylcholine. In another aspect, the carrier is a cationic polymer, for example polyethyleneimine, polyamine, or polyvinylamine.

A significant advance in delivery formulations has been an increased understanding of the way helper components influence the efficiency of formulations. Helper components can include chemical structures added to the primary drug delivery system. Often, helper components can improve particle stability or delivery to a specific organ. For example, nanoparticles can be made of lipids, but the delivery mediated by these lipid nanoparticles can be affected by the presence of hydrophilic polymers and/or hydrophobic molecules. One important hydrophilic polymer that influences nanoparticle delivery is poly(ethylene glycol). Other hydrophilic polymers include non-ionic surfactants. Hydrophobic molecules that affect nanoparticle delivery include cholesterol, 1-2-Distearoyl-sn-glyerco-3-phosphocholine (DSPC), 1-2-di-O-octadecenyl-3-trimethylammonium propane (DOTMA), 1,2-dioleoyl-3-trimethylammonium-propane (DOTAP), and others.

In some aspects of the disclosure, the multi-conjugates can be encapsulated in exosomes. Exosomes are cell-derived vesicles having diameters between 30 and 100 nm that are present in biological fluids, including blood, urine, and cultured medium of cell cultures. Exosomes, including synthetic exosomes and exosome mimetics, can be adapted for use in drug delivery according to the skill in the art. See, e.g., “A comprehensive overview of exosomes as drug delivery vehicles—endogenous nanocarriers for targeted cancer therapy” Biochim Biophys Acta. 1846(1):75-87 (2014); “Exosomes as therapeutic drug carriers and delivery vehicles across biological membranes: current perspectives and future challenges” Acta Pharmaceutica Sinica B, Available online 8 Mar. 2016 (In Press); and “Exosome mimetics: a novel class of drug delivery systems” International Journal of Nanomedicine, 7: 1525-1541 (2012).

In some aspects of the disclosure, the multi-conjugates can be encapsulated in microvesicles. Microvesicles (sometimes called, circulating microvesicles, or microparticles) are fragments of plasma membrane ranging from 100 nm to 1000 nm shed from almost all cell types and are distinct from smaller intracellularly generated extracellular vesicles known as exosomes. Microvesicles play a role in intercellular communication and can transport mRNA, miRNA, and proteins between cells. Microvesicles, including synthetic microvesicles and microvesicle mimetics, can be adapted for use in drug delivery according to the skill in the art. See, e.g., “Microvesicle- and exosome-mediated drug delivery enhances the cytotoxicity of Paclitaxel in autologous prostate cancer cells” Journal of Controlled Release, 220: 727-737 (2015); “Therapeutic Uses of Exosomes” J Circ Biomark, 1:0 (2013).

In some aspects of the disclosure, the multi-conjugates can be delivered using viral vectors. Viral vectors are tools commonly used by molecular biologists to deliver genetic material into cells. This process can be performed inside a living organism (in vivo) or in cell culture (in vitro). Viral vectors can be adapted for use in drug delivery according to the skill in the art. See, e.g., “Viruses as nanomaterials for drug delivery” Methods Mol Biol, 26: 207-21 (2011); “Viral and nonviral delivery systems for gene delivery” Adv Biomed Res, 1:27 (2012); and “Biological Gene Delivery Vehicles: Beyond Viral Vectors” Molecular Therapy, 17(5): 767-777 (2009).

One skilled in the art will appreciate that known delivery formulations, vehicles and targeting moieties can generally be adapted for use according to the present disclosure. Relevant teachings and examples are disclosed in U.S. Pat. Nos. 9,644,209 and 10,597,659; WO 2016/205410 A2; WO 2018/145086 A1; and WO 2020/180897, each of which are incorporated herein by reference in their entirety.

General procedures for oligonucleotide synthesis, annealing conditions, lipid nanoparticle formulation and characterization, preparation of a functionalized oligonucleotides, preparation of DTME mono-substituted with an oligonucleotide, the synthesis and formulation of multimeric oligonucleotides, including attaching cell-targeting moieties to said multimeric oligonucleotides, protocols for animal experiments, including the measurement of serum half-life and gene knock down are described in detail in WO2016/205410, WO2018/145086, and WO 2020/180897, each of which is incorporated herein by reference.

The following Examples are illustrative and not restrictive. Many variations of the technology will become apparent to those of skill in the art upon review of this disclosure. The scope of the technology should, therefore, be determined not with reference to the Examples, but instead should be determined with reference to the appended claims along with their full scope of equivalents.

### EXAMPLES

### Peptide Compounds

### Example 1: Mono-Substitution of DTME with a Peptide

The transduction domain of HIV-1TAT protein (YGRKKRRQRRR) is prepared by solid phase synthesis with a C-terminal cysteine residue. After purification the end product is dissolved in aqueous dimethylformamide and treated with a forty-fold excess of dithiobismaleimidoethane (DTME) and the resulting mixture agitated at room temperature. On completion of the reaction the desired DTME-mono-peptide product is isolated by preparative ion exchange chromatography.

### Example 2: Mono-Substitution of a Nucleotide-Based Homo-Bivalent Linker with a Peptide

The transduction domain of HIV-1TAT protein (YGRKKRRQRRR) prepared by solid phase synthesis with a C-terminal cysteine residue (as in Example 1) is dissolved in aqueous dimethylformamide and treated with a forty-fold excess of 5′-O-maleimidoethyl-thymidylylthymidine-3′-O-ethylmaleimide (MTTM) and the resulting mixture agitated at room temperature. On completion of the reaction the desired MTTM-mono-peptide product is isolated by preparative ion exchange chromatography.

### Example 3: Peptide-siRNA Conjugate

The DTME-mono-(YGRKKRRQRRR) peptide product prepared in Example 1 is dissolved in water and treated with a terminally thiolated siRNA targeting Transthyretin (TTR) mRNA. The resulting peptide-DTME-TTR conjugate is isolated and purified by chromatography.

### Organometallic Compounds

### Example 4: Mono-Substitution of DTME with an Organometallic Compound

6-mercaptohexanoic acid is dissolved in aqueous acetonitrile and treated with a forty-fold excess of DTME. The resulting DTME-mono-thiohexanoic acid product is isolated by preparative hplc and dissolved in anhydrous tetrahydrofuran. Dicyclohexyl carbodiimide is added and the mixture stirred for one hour. Ferrocenylmethyl alcohol is added and the whole stirred at room temperature for 2-3 hours. After workup, the desired ferrocenylmethyl DTME-mono-thiohexanoate is isolated by silica gel chromatography.

### Example 5: Mono-Substitution of a Nucleotide-Based Homo-Bivalent Linker with an Organometallic Compound

6-mercaptohexanoic acid is dissolved in aqueous dimethylformamide and treated with a forty-fold excess of MTTM. The resulting MTTM-mono-thiohexanoic acid product is isolated by preparative hplc and dissolved in anhydrous tetrahydrofuran. Dicyclohexyl carbodiimide is added and the mixture stirred for one hour. Ferrocenylmethyl alcohol is added and the whole stirred at room temperature for 2-3 hours. After workup, the desired ferrocenylmethyl MTTM-mono-thiohexanoate is isolated by silica gel chromatography.

### Example 6: Mono-Substitution of a Peptide-Based Homo-Bivalent Linker with an Organometallic Compound

6-mercaptohexanoic acid is dissolved in aqueous dimethylformamide and treated with a forty-fold excess of N, N, bis-(6-maleimidohexanoyl) glycine-lysine (MGKM). The resulting MGKM-mono-thiohexanoic acid product is isolated by preparative hplc and dissolved in anhydrous tetrahydrofuran. Dicyclohexyl carbodiimide is added and the mixture stirred for one hour. Ferrocenylmethyl alcohol is added and the whole stirred at room temperature for 2-3 hours. After workup, the desired ferrocenylmethyl MGKM-mono-thiohexanoate is isolated by silica gel chromatography.

### Example 7: Peptide-Organometallic Conjugate

The transduction domain of HIV-1TAT protein (YGRKKRRQRRR) prepared by solid phase synthesis with a C-terminal cysteine residue in Example 1 is dissolved in dimethylformamide and treated with the ferrocenylmethyl MGKM-mono-thiohexanoate derivative prepared in Example 6. The resulting peptide-MGKM-thiohexanoate is isolated by chromatography.

### Antibody Compounds

### Example 8: Mono-Substitution of DTME with an Antibody Fragment

An antibody single chain FV fragment carrying a free cysteine is expressed in E. Coli (Kipriyanov, S M et al; Molecular Immunology, 31, No 14, 1047-1058 (1994)). The antibody is dissolved in aqueous dimethylformamide and treated with a forty-fold excess of dithiobismaleimidoethane (DTME) and the resulting mixture agitated at room temperature. On completion of the reaction the desired DTME-mono-antibody product is isolated by preparative ion exchange chromatography.

### Example 9: Mono-Substitution of a Nucleotide-Based Homo-Bivalent Linker with an Antibody Fragment

The antibody single chain FV fragment carrying a free cysteine prepared in Example 8 is dissolved in aqueous dimethylformamide and treated with a forty-fold excess of MTTM and the resulting mixture agitated at room temperature. On completion of the reaction the desired MTTM-mono-single chain FV product is isolated by preparative chromatography.

### Example 10: Antibody Fragment-siRNA Conjugate

The DTME-mono-single chain FV product produced in Example 8 is dissolved in water and treated with a terminally thiolated siRNA targeting myotonic dystrophy protein kinase (DMPK) mRNA. The resulting FV-DTME-TTR conjugate is isolated and purified by chromatography.

### Small Molecule Compounds

### Example 11: Mono-Substitution of DTME with a Small Molecule

6-mercaptohexanoic acid is dissolved in aqueous acetonitrile and treated with a forty-fold excess of DTME. The resulting DTME-mono-thiohexanoic acid product is isolated by preparative hplc and dissolved in anhydrous dimethylformamide. N-hydroxysuccinimide is added and the mixture stirred for one hour. Lenalidomide is added and the whole stirred at room temperature for 2-3 hours. After workup, the desired DTME-mono-thiohexamido-lenalidomide is isolated by silica gel chromatography.

### Example 12: Mono-Substitution of a Nucleotide-Based Homo-Bivalent Linker with a Small Molecule

6-mercaptohexanoic acid is dissolved in aqueous dimethylformamide and treated with a forty-fold excess of MTTM. The resulting MTTM-mono-thiohexanoic acid product is isolated by preparative hplc and dissolved in anhydrous dimethylformamide. N-hydroxysuccinimide is added and the mixture stirred for one hour. Lenalidomide is added and the whole stirred at room temperature for 2-3 hours. After workup, the desired MTTM-mono-thiohexamido-lenalidomide is isolated by silica gel chromatography.

### Example 13: Antibody Fragment-Small Molecule Conjugate

The antibody single chain FV fragment carrying a free cysteine prepared in Example 8 is dissolved in aqueous dimethylformamide and added to a solution of the MTTM-mono-thiohexamido-lenalidomide prepared in Example 12. On completion of the reaction the desired single chain FV-MTTM-thiohexamido-lenalidomide product is isolated by preparative chromatography.

1. A compound comprising a homo-bivalent covalent linker substituted on one end by a substituent X, wherein X comprises a biological moiety other than a nucleic acid, wherein the other end of the homo-bivalent linker is unsubstituted, and wherein the compound is at least 75% pure.

2. The compound of claim 1, wherein the compound comprises Structure 1:
X—R1-R2-A-R3-B (Structure 1)
wherein:
X is a substituent comprising a biological moiety other than a nucleic acid;
R1 is a group comprising phosphodiester, thiophosphodiester, sulfate, amide, triazole, heteroaryl, ester, ether, thioether, disulfide, thiopropionate, acetal, glycol, or is absent;
R2 is a spacer group, or is absent;
A is a group comprising the reaction product of a first nucleophile and a first electrophile;
R3 is a group comprising a C₂-C₁₀ alkyl, C₂-C₁₀ alkoxy, C₁-C₁₀ aryl, C₂-C₁₀ alkyldithio, amide, ether, thioether, ester, oligonucleotide, oligopeptide, thiopropionate, or disulfide; and
B is a group comprising a second nucleophile or a second electrophile, wherein the second nucleophile is the same as the first nucleophile, and the second electrophile is the same as the first electrophile.

3. The compound of claim 2, wherein R2 comprises a C₂-C₁₀ alkyl, C₂-C₁₀ alkoxy, or C₁-C₁₀ aryl, or is absent.

4. The compound of claim 2, wherein the first nucleophile and first electrophile of A comprise (i) a thiol and a maleimide, optionally wherein the reaction product of the thiol and maleimide is a derivative of succinamic acid; (ii) a thiol and a vinylsulfone; (iii) a thiol and a pyridyldisulfide; (iv) a thiol and an iodoacetamide; (v) a thiol and an acrylate; (vi) an azide and an alkyne; or (vii) an amine and a carboxyl.

5. The compound of claim 4, wherein A is a group comprising the reaction product of a thiol and a maleimide, optionally wherein the reaction product of the thiol and maleimide is a derivative of succinamic acid.

6. The compound of claim 2, wherein R3 is a group comprising a thiopropionate or disulfide, an oligonucleotide, or an oligopeptide.

7. The compound of claim 2, wherein the compound comprises Structure 2 or a pyrrolidinedione ring-opened derivative thereof, optionally wherein the pyrrolidinedione ring-opened derivative of Structure 2 is a derivative of succinamic acid:

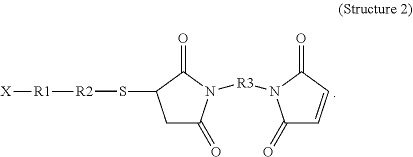

8. The compound of claim 7, wherein R2 is a spacer group comprising a C₂-C₁₀ alkyl, C₂-C₁₀ alkoxy, or C₁-C₁₀ aryl.

9. The compound of claim 7, wherein:
X is a peptide or protein, or a derivative thereof;
R1 and R2 are absent; and
R3 is a group comprising a thiopropionate, disulfide, or oligonucleotide.

10. The compound of claim 7, wherein:
X is an organometallic compound, or a derivative thereof;
R1 is an ester group;
R2 is a spacer group comprising a C₂-C₁₀ alkyl; and
R3 is a group comprising a thiopropionate, a disulfide, an oligonucleotide, or an oligopeptide.

11. The compound of claim 7, wherein:
X is a small molecule, or a derivative thereof;
R1 is an ester group;
R2 is a spacer group comprising a C₂-C₁₀ alkyl; and
R3 is a group comprising a thiopropionate, a disulfide, an oligonucleotide, or an oligopeptide.

12. The compound of claim 1, wherein the homo-bivalent covalent linker comprises a linker that is cleavable under intracellular conditions.

13. The compound of claim 2, wherein the R3 group comprises a linker that is cleavable under intracellular conditions.

14. The compound of claim 2, wherein the substituent X is a peptide, protein, lipid, carbohydrate, carboxylic acid, vitamin, steroid, lignin, small molecule, organometallic compound, or a derivative of any of the foregoing.

15. The compound of claim 14, wherein the substituent X is a peptide, or a derivative thereof.

16. The compound of claim 14, wherein the peptide is the transduction domain of HIV-1TAT protein, a Centyrin, a constrained peptide, a pHLIP peptide, or derivatives thereof.

17. The compound of claim 14, wherein the substituent X is an antibody or antibody fragment, or a derivative thereof.

18. The compound of claim 17, wherein the antibody fragment is a single-chain variable fragment, or derivative thereof.

19. The compound of claim 14, wherein the substituent X is a carbohydrate, or a derivative thereof.

20. The compound of claim 14, wherein the substituent X is a fatty acid, or a derivative thereof.

21. The compound of claim 14, wherein the substituent X is a vitamin, or a derivative thereof.

22. The compound of claim 21, wherein the substituent X is tocopherol or folate, or a derivative thereof.

23. The compound of claim 14, wherein the substituent X is a cholesterol, or a derivative thereof.

24. The compound of claim 14, wherein the substituent X is (2S,2′S)-2,2′-(Carbonyldiimino)dipentanedioic acid (DUPA), or a derivative thereof.

25. The compound of claim 14, wherein the substituent X is anisamide, or a derivative thereof.

26. The compound of claim 14, wherein the substituent X is an organometallic compound, or a derivative thereof.

27. The compound of claim 26, wherein the organometallic compound is ferrocene, or a derivative thereof.

28. The compound of claim 14, wherein substituent X is a small molecule, or a derivative thereof.

29. The compound of claim 28, wherein the small molecule is lenalidomide, or a derivative thereof.

30. The compound of claim 2, where in the compound is at least 80, 85, 90, 95, 96, 97, 98, 99, or 100% pure.

31-109. (canceled)
